(19) United States
(12) Patent Application Publication
(10) Pub. No.: US 2023/0364026 A1
(43) Pub. Date: Nov. 16, 2023

(54) STABILIZED SOLID NANOPARTICLE FORMULATIONS OF CANNABINOIDS AND CANNABINOID ANALOGS WITH REDUCED OSTWALD RIPENING FOR ORAL, INHALATION, NASAL AND PARENTERAL DRUG DELIVERY

(71) Applicant: Luminus Biosciences, Inc., San Antoni, CA (US)

(72) Inventors: Ulagaraj Selvaraj, San Antonio, TX (US); David L. Woody, San Antonio, TX (US); John H. Boatright, San Antonio, TX (US); Dong Wen, San Antonio, TX (US)

(21) Appl. No.: 18/027,014

(22) PCT Filed: Sep. 17, 2021
(86) PCT No.: PCT/US2021/050984

§ 371 Date: Mar. 17, 2023

Related U.S. Application Data
(60) Provisional application No. 63/079,795, filed on Sep. 17, 2020.

(51) Int. Cl. A61K 9/51; A61K 31/00

(52) U.S. Cl. CPC A61K 9/5169; A61K 31/658; A61K 9/5123; B82Y 5/00

(57) ABSTRACT

The present invention provides compositions comprising cannabinoids and/or cannabinoid analogs and processes for producing the same.

### FIELD OF THE INVENTION

The present invention belongs to the fields of pharmacology, medicine and medicinal chemistry.

### BACKGROUND OF THE INVENTION

The therapeutic efficacy of most cannabinoid-based drugs is predicated on achieving adequate local delivery to the target sites. Inadequate specific delivery can lead to the frequently low therapeutic index seen with current cannabinoids. This translates into significant systemic toxicities attributable to the wide dissemination and nonspecific action of many of these compounds (Natascia Bruni, et al., 2018).

Another problem is the solubility of some of the potent therapeutic agents in suitable pharmaceutically acceptable vehicle for administration. The therapeutic class of agents include epilepsy/seizure, pain, Alzheimer's, anorexia, anxiety, atherosclerosis, arthritis cancer, colitis/Crohn's, depression, diabetes, fibromyalgia, glaucoma, irritable bowel, multiple sclerosis, neurodegeneration, obesity, osteoporosis, Parkinson's, PTSD, schizophrenia, substance dependence/addiction, and stroke/traumatic brain injury.

However, it is now known as a fact that these important classes of drugs have been formulated in vehicles which are very toxic to humans.

### Cannabinoids and Cannabinoid Analogs

Examples of cannabinoids and cannabinoid analogs include plant derived tetrahydrocannabidiol (THC), synthetic tetrahydrocannabinol (THC or Dronabinol), cannabidiol (CBD), cannabinol (CBN), cannabigerol (CBG), tetrahydrocannabinolic acid (THCA), cannabidivarine (CBDV), nabilone, HU-210, and dexanabinol.

THC is the primary psychoactive ingredient, and cannabidiol (CBD) is the major non-psychoactive ingredient in cannabis. THC binds to two G-protein-coupled cell membrane receptors, therefore named the cannabinoid type 1 (CB₁) and type 2 (CB₂) receptors, to exert its effects. CB₁ receptors are found primarily in the brain but also in several peripheral tissues. CB₂ receptors can be found on immune cells, inflammatory cells, and cancer cells.

The human body produces substances called endocannabinoids that act on CB₁ and CB₂ receptors but are chemically different from THC and some other plant cannabinoids that also act on CB₁ and CB₂ receptors. The endocannabinoid system is widely distributed throughout the body, acting to regulate the activity of various kinds of cells and tissue. Since the endocannabinoid system is so widely distributed throughout the body, cannabinoids can cause many changes in body functions.

Unlike THC, CBD has the little affinity for the CB₁ and CB₂ receptors but acts as an indirect antagonist of these receptors. CBD modulates the effect of THC, and both THC and CBD are antioxidants, inhibiting NMDA-mediated excitotoxicity under conditions of traumatic head injury, stroke and degenerative brain diseases. CBD also stimulates vanilloid pain receptors (VR1), inhibits uptake of the anandamide, and weakly inhibits its breakdown. These findings have important implications in elucidating the pain-relieving, anti-inflammatory, and immunomodulatory effects of CBD. The combination of THC and CBD produces therapeutic benefits that are greater than the individual components.

Dronabinol, the active ingredient in MARINOL® Capsules, is synthetic delta-9-tetrahydrocannabinol (delta-9-THC). Delta-9-tetrahydrocannabinol is also a naturally occurring component of Cannabis sativa L. (Marijuana). Dronabinol is a light-yellow resinous oil that is sticky at room temperature and hardens upon refrigeration. Dronabinol is insoluble in water and is formulated in sesame oil. It has a pKa of 10.6 and an octanol-water partition coefficient: 6,000:1 at pH 7 (Whiting, P. F., et al., 2015).

Dronabinol (Marinol®), contains the trans isomer of THC dissolved in sesame oil contained within a gelatin capsule. Marinol® capsules contain 2.5, 5, or 10 mg of dronabinol. This drug is approved by the FDA approved for two indications: 1) chemotherapy-induced nausea and vomiting (CINV), and 2) anorexia associated with weight loss in patients with the acquired immunodeficiency syndrome (Walther S, et al., 2006).

Marinol® does not contain any actual plant cannabinoids. The important main difference between dronabinol and THC is the origin of their existence. Dronabinol is human-made and manufactured in a laboratory, while the actual THC cannabinoid is produced naturally by the cannabis plant

Unimed Pharmaceuticals, a subsidiary of Solvay Pharmaceuticals, was initially granted approval in 1985 for Marinol® in a fixed-dose pill form for nausea. In 1992, appetite stimulation was added to its indications. It was classified as a Schedule I drug until it was moved to Schedule III in 1999. Marinol® is manufactured by Patheon Softgels, Inc., for Abbvie Inc., and prescribed for management of appetite loss associated with weight loss in acquired immune deficiency syndrome (AIDS), and nausea and vomiting related to cancer chemotherapy in patients who have failed to respond adequately to conventional treatments to relieve nausea and vomiting.

In 2016 the FDA approved a new liquid formulation of dronabinol. The updated version of the drug is made by DPT Lakewood LLC for Insys Therapeutics and is marketed under the brand name Syndros®.

Indications are the same for Syndros® as they are for Marinol: anorexia associated with weight loss in patients with AIDS, and nausea and vomiting associated with cancer chemotherapy in patients who have failed to respond adequately to conventional treatment.

Cesamet® is the brand name for nabilone. Nabilone is a purely human-made synthetic drug. Nabilone is a potent cannabinoid agonist, having an affinity of 2.2 nM for human CB₁ receptors and 1.8 nM for human CB₂ receptors. The activation of CB1 reduces pro-emetic signaling in the vomiting center, thus inhibiting nausea and vomiting. Cesamet claims it replicates the healing properties of THC, but does not actually contain any of the constituents found in the Cannabis plant and thus, cannot tap into the entourage effect produced by whole plant cannabis medicines.

Cesamet® is classified as an antiemetic. Antiemetics are medicines that help prevent or treat chemotherapy-induced nausea and vomiting (CINV). Cesamet® is to be prescribed to people who continue to experience these symptoms after trying other traditional medications, specifically antiemetics, to find relief.

Nabilone is an orally active, human-made synthetic cannabinoid. In its raw form, nabilone is a white to off-white polymorphic crystalline powder. When dissolved in water, the solubility of nabilone is less than 0.5 mg/L, with pH values ranging from 1.2 to 7.0. Nabilone is (±)-trans-3-(1,1-dimethylheptyl)-6,6a,7,8,10,10a-hexahydro-1-hydroxy-6-6-dimethyl-9H-dibenzo[b,d] pyran-9-one and has the empirical formula C₂₄H₃₆O₃. It has a molecular weight of 372.55.

A 1 mg Cesamet® capsule contains 1 mg of nabilone and the inactive ingredients: povidone and corn starch. Povidone is used in the pharmaceutical industry as a synthetic polymer vehicle for dispersing and suspending drugs. When administered orally, nabilone appears to be completely absorbed from the human gastrointestinal tract.

Another cannabinoid pharmaceutical of note is Nabiximols (Sativex®), which is a whole-plant extract of marijuana, and contains THC and CBD in a 1.08:1.00 ratio. It is administered as an oral mucosal spray (Russo E B et al., 2007). In Canada, Sativex® is approved for the relief of neuropathic pain (pain due to disease of the nervous system), pain and spasticity (muscular stiffness) due to multiple sclerosis, and of severe pain due to advanced cancer. Sativex® is undergoing clinical trials in the United States and is available on a limited basis by prescription in the United Kingdom and Spain.

Many case reports and interviews of parents indicated that up to 70% of the children treated had a 50% or greater reduction in seizure frequency. These encouraging observations have led to the initiation of properly designed clinical trials with a cannabis extract containing 99% pure CBD (Epidiolex®) for the treatment of diverse types of childhood epilepsy. The FDA approved Epidiolex® oral solution in 2018 for the treatment of seizures associated with Lennox-Gastaut syndrome (LGS) or Dravet syndrome in patients two years of age or older.

Synthetic cannabinoid drugs, which originate from four chemically distinct groups: (i) the JWH compounds, synthesized by John W. Huffman (JWH) in the 1980s, of which JWH-018 is the most studied and best characterized to date; (ii) the CP-compounds, a cyclohexylphenol series synthesized by Pfizer in the 1970s, with the identified CP-47,497 and its modified version CP-47,497-C8 (obtained by extending the dimethylheptyl side chain to dimethyloctyl) (Huffman J. W. et al.: 2008); (iii) the HU-compounds, synthesized in the 1960s at the Hebrew University; and (iv) the benzoylindoles, such as AM-694 and RCS-4 (EMCDDA, 2009). Both JWH-018 and CP 47,497-C8 act as agonists at CB1 receptor and, therefore, produce cannabis-like effects. Due to their high pharmacological potency in vitro, it is likely that relatively low doses are sufficient for activity. The duration of effects in humans compared to THC seems to be shorter for JWH-018 (1-2 hours) and considerably longer for CP 47,497-C8 (5-6 hours), as reported in a self-experiment (Auwärter et al., 2009).

HU210, a synthetic analogue of THC, with high lipophilicity has been evaluated (Howlett A. C. et al.: 2002). The efficacy of HU210 at both C_{B1} and CB₂ receptors is like that of other cannabinoids; however, the affinity of HU210 for these receptors is higher. This results in HU210 being a potent cannabinoid agonist with long-lasting pharmacological effects in vivo.

HU-211, the full chemical name of which is 1,1-dimethylheptyl-(3S,4S-7-hydroxy-Δ⁶-tetrahydrocannabinol, was disclosed in U.S. Pat. No. 4,876,276 and subsequently assigned the trivial chemical name dexanabinol (CAS number: 112-924-45-5). At first, potential therapeutic applications of dexanabinol included known attributes of marijuana itself such as anti-emesis, analgesia, and anti-glaucoma, as disclosed in U.S. Pat. No. 4,876,276.

It was later established that novel synthetic compounds could block the NMDA receptor, as disclosed in U.S. Pat. Nos. 5,284,867, 5,521,215 and 6,096,740.

Dexanabinol and its analogues appear to share anti-oxidative, immunomodulatory and anti-inflammatory properties in addition to their capacity to block the NMDA receptor, as disclosed in U.S. Pat. Nos. 5,932,610, 6,331,560 and 6,545,041. 5,284,867.

### Method for Nanoparticle Preparation:

There are several methods disclosed in the literature for the preparation of solid nanoparticles. For example, solid lipid nanoparticles (SLN) are nanoparticles with a matrix being composed of a solid lipid, i.e. the lipid is solid at room temperature and at body temperature (Muller, R H, et al.: 2000). The lipid is melted approximately 5° C. above its melting point and the drug dissolved or dispersed in the melted lipid. Subsequently, the melt is dispersed in a hot surfactant solution by high speed stirring. The coarse emulsion obtained is homogenised in a high-pressure unit, typically at 500 bar and three homogenisation cycles. A hot oil-in-water nanoemulsion is obtained, cooled, the lipid recrystallises and forms solid lipid nanoparticles. Identical to the drug nanocrystals the SLN possess adhesive properties. They adhere to the gut wall and release the drug exactly where it should be absorbed. In addition, the lipids are known to have absorption promoting properties, not only for lipophilic drugs such as Vitamin E but also drugs in general (Porter C J and Charman W N: 2001). There are even differences in the lipid absorption enhancement depending on the structure of the lipids (Sek L, et al.: 2002). Basically, the body is taking up the lipid and the solubilised drug at the same time.

Meanwhile the second generation of lipid nanoparticles with solid matrix has been developed, the so-called nanostructured lipid carriers. The NLC® are characterised that a certain nanostructure is given to their particle matrix by preparing the lipid matrix from a blend of a solid lipid with a liquid lipid (oil). The mixture is still solid at 40° C. These particles have improved properties regarding payload of drugs, more flexibility in modulating the drug release profile and being also suitable to trigger drug release (Muller, R. H., et al.: 2002). They can also be used for oral and parenteral drug administration identical to SLN but have some additional interesting features.

In the Lipid Drug Conjugate (LDC®) nanoparticle technology (Olbricha C, et al.: 2004), the “conjugates” (term used in its broadest sense) were prepared either by salt formation (e.g. amino group containing molecule with fatty acid) or alternatively by covalent linkage (e.g. ether, ester, e.g. tributyrin). Most of the lipid conjugates melt somewhere about approximately 50-100° C. The conjugates are melted and dispersed in a hot surfactant solution. Further processing was performed identical to SLN and NLC. The obtained emulsion system is homogenised by high-pressure homogenisation, the obtained nanodispersion cooled, the conjugate recrystallises and forms LDC nanoparticles. One could consider this suspension also as a nanosuspension of a pro-drug.

The common method for the preparation of solid nanoparticles is by the solvent evaporation of an oil-in-water emulsion. The oil-phase contains one or more pharmaceutical substances and the aqueous phase contains just the buffering materials or an emulsifier. An emulsion consists of two immiscible liquids (usually oil and water), with one of the liquids dispersed as small spherical droplets in the other. In most foods, for example, the diameters of the droplets usually lie somewhere between 0.1 and 100 m. An emulsion can be conveniently classified according to the distribution of the oil and aqueous phases. A system that consists of oil droplets dispersed in an aqueous phase is called an oil-in-water or O/W emulsion (e.g, mayonnaise, milk, cream etc.). A system that consists of water droplets dispersed in an oil phase is called a water-in-oil or W/O emulsion (e.g. margarine, butter and spreads). The process of converting two separate immiscible liquids into an emulsion, or of reducing the size of the droplets in a preexisting emulsion, is known as homogenization.

It is possible to form an emulsion by homogenizing pure oil and pure water together, but the two phases rapidly separate into a system that consists of a layer of oil (lower density) on top of a layer of water (higher density). This is because droplets tend to merge with their neighbors, which eventually leads to complete phase separation. Emulsions usually are thermodynamically unstable systems. It is possible to form emulsions that are kinetically stable (metastable) for a reasonable period (a few minutes, hours, days, weeks, months, or years) by including substances known as emulsifiers and/or thickening agent prior to homogenization.

Emulsifiers are surface-active molecules that adsorb to the surface of freshly formed droplets during homogenization, forming a protective membrane that prevents the droplets from coming close enough together to aggregate. Most emulsifiers are molecules having polar and nonpolar regions in the same molecule. The most common emulsifiers used in the food industry are amphiphilic proteins, small-molecule surfactants, and monoglycerides, such as sucrose esters of fatty acids, citric acid esters of monodiglycerides, salts of fatty acids, etc (Krog J. N., 1990).

Thickening agents are ingredients that are used to increase the viscosity of the continuous phase of emulsions and they enhance emulsion stability by retarding the movement of the droplets. A stabilizer is any ingredient that can be used to enhance the stability of an emulsion and may therefore be either an emulsifier or thickening agent.

The term “emulsion stability” is broadly used to describe the ability of an emulsion to resist changes in its properties with time (McClements D. J., 2007). Emulsions may become unstable through a variety of physical processes including creaming, sedimentation, flocculation, coalescence, and phase inversion. Creaming and sedimentation are both forms of gravitational separation. Creaming describes the upward movement of droplets because they have a lower density than the surrounding liquid, whereas sedimentation describes the downward movement of droplets due to the fact that they have a higher density than the surrounding liquid. Flocculation and coalescence are both types of droplet aggregation. Flocculation occurs when two or more droplets come together to form an aggregate in which the droplets retain their individual integrity, whereas coalescence is the process where two or more droplets merge together to form a single larger droplet. Extensive droplet coalescence can eventually lead to the formation of a separate layer of oil on top of a sample, which is known as “oiling off”.

Most emulsions can conveniently be considered to consist of three regions that have different physicochemical properties: the interior of the droplets, the continuous phase, and the interface. The molecules in an emulsion distribute themselves among these three regions according to their concentration and polarity (Wedzicha B. L., 1988). Nonpolar molecules tend to be located primarily in the oil phase, polar molecules in the aqueous phase, and amphiphilic molecules at the interface. It should be noted that even at equilibrium, there is a continuous exchange of molecules between the different regions, which occurs at a rate that depends on the mass transport of the molecules through the system. Molecules may also move from one region to another when there is some alteration in the environmental conditions of an emulsion (e.g, a change in temperature or dilution within the mouth). The location and mass transport of the molecules within an emulsion have a significant influence on the aroma, flavor release, texture, and physicochemical stability of food products (Wedzicha B L, et al., 1991).

Many properties of the emulsions can only be understood with reference to their dynamic nature. The formation of emulsions by homogenization is a highly dynamic process which involves the violent disruption of droplets and the rapid movement of surface-active molecules from the bulk liquids to the interfacial region. Even after their formation, the droplets in an emulsion are in continual motion and frequently collide with one another because of their Brownian motion, gravity, or applied mechanical forces (Dukhin A. S., and Dukhin S. S., 2014). The continual movement and interactions of droplets cause the properties of emulsions to evolve over time due to the various destabilization processes such as change in temperature or in time.

The most important properties of emulsion are determined by the size of the droplets they contain. Consequently, it is important to control, predict and measure, the size of the droplets in emulsions. If all the droplets in an emulsion are of the same size, the emulsion is referred to as monodisperse, but if there is a range of sizes present, the emulsion is referred to as polydisperse. The size of the droplets in a monodisperse emulsion can be completely characterized by a single number, such as the droplet diameter (d) or radius (r). Monodisperse emulsions are sometimes used for fundamental studies because the interpretation of experimental measurements is much simpler than that of polydisperse emulsions. Nevertheless, emulsions by homogenization always contain a distribution of droplet sizes, and so the specification of their droplet size is more complicated than that of monodisperse systems. Ideally, one would like to have information about the full particle size distribution of an emulsion (i.e, the size of each of the droplets in the system). In many situations, knowledge of the average size of the droplets and the width of the distribution is sufficient (Hunter R J: 1986).

An efficient emulsifier produces an emulsion in which there is no visible separation of the oil and water phases over time. Phase separation may not become visible to the human eye for a long time, even though some emulsion breakdown has occurred. A more quantitative method of determining emulsifier efficiency is to measure the change in the particle size distribution of an emulsion with time. An efficient emulsifier produces emulsions in which the particle size distribution does not change over time, whereas a poor emulsifier produces emulsions in which the particle size increases due to coalescence and/or flocculation. The kinetics of emulsion stability can be established by measuring the rate at which the particle size increases with time.

### Proteins as Emulsifiers:

In oil-in-water emulsions, proteins are used mostly as surface active agents and emulsifiers. One of the food proteins used in o/w emulsions is whey proteins. The whey proteins include four proteins: β-lactoglobulin, α-lactalbumin, bovine serum albumin and immunoglobulin (Tornberg E, et al.: 1990). Commercially, whey protein isolates (WPI) with isolectric point ˜5 are used for o/w emulsion preparation. According to Hunt (Hunt J. A., and Dalgleish D G: 1995), whey protein concentrations of 8% have been used to produce self-supporting gels. Later, the limiting concentrations of whey protein to produce self-supporting gels are known to be reduced to 4-5%. It is possible to produce gels at whey protein concentrations as low as 2% w/w, using heat treatments at 90° C. or 121° C. and ionic strength in excess of 50 mM.

U.S. Pat. No. 6,106,855 discloses a method for preparing stable oil-in-water emulsions by mixing oil, water and an insoluble protein at high shear. By varying the amount of insoluble protein, the emulsions may be made liquid, semisolid or solid. The preferred insoluble proteins are insoluble fibrous proteins such as collagen. The emulsions may be medicated with hydrophilic or hydrophobic pharmacologically active agents and are useful as or in wound dressings or ointments.

U.S. Pat. No. 6,616,917 discloses an invention relating to a transparent or translucent cosmetic emulsion comprising an aqueous phase, a fatty phase and a surfactant, the said fatty phase containing a miscible mixture of at least one cosmetic oil and of at least one volatile fluoro compound, the latter compound being present in a proportion such that the refractive index of the fatty phase is equal to ±0.05 of that of the aqueous phase. The invention also relates to the process for preparing the emulsion and the use of the emulsion in skincare, hair conditioning and antisun protection and/or artificial tanning.

Proteins derived from whey are widely used as emulsifiers (Dalgleish D. G., 1996). They adsorb to the surface of oil droplets during homogenization and form a protective membrane, which prevents droplets from coalescing (Dickinson E., 1998). The physicochemical properties of emulsions stabilized by whey protein isolates (WPI) are related to the aqueous phase composition (e.g, ionic strength and pH) and the processing and storage conditions of the product (e.g, heating, cooling, and mechanical agitation). Emulsions are prone to flocculation around the isoelectric point of the WPI but are stable at higher or lower pH. The stability to flocculation could be interpreted in terms of colloidal interactions between droplets, i.e, van der Waals, electrostatic repulsion and steric forces. The van der Waals interactions are short-range due to their dependence on the inverse 6^{th} power of the distance. Electrostatic interactions between similarly charged droplets are repulsive, and their magnitude and range decrease with increasing ionic strength. Short-range interactions become important at droplet separations of the order of the thickness of the interfacial layer or less, e.g, steric, thermal fluctuation and hydration forces (Israelachvili J N: 1992). Such interactions are negligible at distances greater than the thickness of the interfacial layer, but become strongly repulsive when the layers overlap, preventing droplets from getting closer. It has been shown that the criteria for the protein emulsifiers appear to be the ability to adsorb quickly at the oil/water interface and surface hydrophobicity is of secondary importance (Mangino M E: 1994).

Thus, in the preparation of nanoparticles using the solvent evaporation technique, proteins can be used as emulisfier to form the fine oil-in-water emulsion and subsequently the organic solvent in the emulsion can be evaporated to form the nanoparticles. Human serum albumin can be ideal for such preparations as it is non-immunogenic in humans, has the desired property as an emulsifier and has preferential targeting property to tumor sites. The measurements using the phosphorescence depolarization technique support a rather rigid heart shaped structure (8 nm×8 nm×3.2 nm) of albumin in a neutral solution of BSA as in the crystal structure of human serum albumin (Ferrer M L, et al.: 2001) and serum albumin have been shown to have good gelling properties.

### Polymers as Emulsifiers:

Apart from proteins as emulsifiers, several natural, semi-natural and synthetic polymers can be used as emulsifiers (Mathur A M, et al.: 1998). The polymer emulsifiers include naturally occurring emulsifiers, for example, agar, carageenan, furcellaran, tamarind seed polysaccharides, gum tare, gum karaya, pectin, xanthan gum, sodium alginate, tragacanth gum, guar gum, locust bean gum, pullulan, jellan gum, gum Arabic and various starches. Semisynthetic emulsifieres include carboxymethyl cellulose (CMC), methyl cellulose (MC), hydroxyethyl cellulose (HEC), alginic acid propylene glycol ester, chemically modified starches including soluble starches, and synthetic polymers including polyvinyl alcohol, polyethylene glycol and sodium polyacrylate. These polymer emulsifiers are used in the production of emulsion compositions such as emulsion flavors or powder compositions such as powder fats and oils and powder flavors. The powder composition is produced by emulsifying an oil, a lipophilic flavor or the like, and an aqueous component with a polymer emulsifier and then subjecting the emulsion to spray drying or the like. In this case, the powder composition is often in the form of a microcapsule.

### Ostwald Ripening:

Generally, if particles with a wide range of sizes are dispersed in a medium there will be a differential rate of dissolution of the particles in the medium. The differential dissolution results in the smaller particles being thermodynamically unstable relative to the larger particles and gives rise to a flux of material from the smaller particles to the larger particles. The effect of this is that the smaller particles dissolve in the medium, whilst the dissolved material is deposited onto the larger particles thereby giving an increase in particle size. One such mechanism for particle growth is known as Ostwald ripening (Ostwald, W., 1897). Ostwald ripening has been studied extensively due to its importance in material and pharmaceutical sciences (Baldan A and Mater J., 2001; Madras G., and McCoy B. J., 2002).

The growth of particles in a dispersion can result in instability of the dispersion during storage, resulting in the sedimentation of particles from the dispersion. It is particularly important that the particle size in a dispersion of a pharmacologically active compound remains constant because a change in particle size is likely to affect the bioavailability, toxicity and hence the efficacy of the compound. Furthermore, if the dispersion is required for intravenous administration, growth of the particles in the dispersion may render the dispersion unsuitable for this purpose, possibly leading to adverse or dangerous side effects.

Theoretically particle growth resulting from Ostwald ripening would be eliminated if all the particles in the dispersion were the same size. However, in practice, it is impossible to achieve a completely uniform particle size and even small differences in particle sizes can give rise to particle growth.

U.S. Pat. No. 4,826,689 describes a process for the preparation of uniform sized particles of a solid by infusing an aqueous precipitating liquid into a solution of the solid in an organic liquid under controlled conditions of temperature and infusion rate, thereby controlling the particle size. U.S. Pat. No. 4,997,454 describes a similar process in which the precipitating liquid is non-aqueous. However, when the particles have a small but finite solubility in the precipitating medium, particle size growth is observed after the particles have been precipitated. To maintain a particle size using these processes it is necessary to isolate the particles as soon as they have been precipitated to minimise particle growth. Therefore, particles prepared according to these processes cannot be stored in a liquid medium as a dispersion. Furthermore, for some materials the rate of Ostwald ripening is so great that it is not practical to isolate small particles (especially nano-particles) from the suspension.

Higuchi and Misra (Higuchi W J and Misra J: 1962) describe a method for inhibiting the growth of the oil droplets in oil-in-water emulsions by adding a hydrophobic compound (such as hexadecane) to the oil phase of the emulsion. U.S. Pat. No. 6,074,986 describes the addition of a polymeric material having a molecular weight of up to 10,000 to the disperse oil phase of an oil-in-water emulsion to inhibit Ostwald ripening. Welin-Berger et al. (Welin-Berger et al. 2000) describe the addition of a hydrophobic material to the oil phase of an oil-in-water emulsion to inhibit Ostwald ripening of the oil droplets in the emulsion. In these latter three references, the material added to the oil phase is dissolved in the oil phase to give a single-phase oil dispersed in the aqueous continuous medium.

EP 589 838 describes the addition of a polymeric stabilizer to stabilize an oil-in-water emulsion wherein the disperse phase is a hydrophobic pesticide dissolved in a hydrophobic solvent.

U.S. Pat. No. 4,348,385 discloses a dispersion of a solid pesticide in an organic solvent to which is added an ionic dispersant to control Ostwald ripening.

WO 99/04766 describes a process for preparing vesicular nano-capsules by forming an oil-in-water emulsion wherein the dispersed oil phase comprises a material designed to form a nano-capsule envelope, an organic solvent and optionally an active ingredient. After formation of a stable emulsion, the solvent is extracted to leave a dispersion of nano-capsules.

U.S. Pat. No. 5,100,591 describes a process in which particles comprising a complex between a water insoluble substance and a phospholipid are prepared by co-precipitation of the substance and phospholipid into an aqueous medium. Generally, the molar ratio of phospholipid to substance is 1:1 to ensure that a complex is formed.

U.S. Pat. No. 4,610,868 describes lipid matrix carriers in which particles of a substance is dispersed in a lipid matrix. The major phase of the lipid matrix carrier comprises a hydrophobic lipid material such as a phospholipid.

One of the inventors has disclosed in U. S. Patent Application Publication No. 2009/0238878 that a substantially stable nanoparticle can be formed by the solvent evaporation of an oil-in-water emulsion using protein such as serum albumin or a polymer such as polyvinyl alcohol as emulsifying agent to inhibit the Ostwald ripening.

What is needed are new compositions and methods of delivering substantially water insoluble cannabinoids and cannabinoid analogs-based drug products in a safe manner to humans who are suffering from various conditions, including epilepsy, pain, nausea and vomiting, cancer and others.

### SUMMARY OF THE INVENTION

The present invention discloses the preparations of substantially stable nanoparticles comprising pharmaceutically active water insoluble substances without appreciable Ostwald ripening. The nanoparticles can be used for the treatment of various conditions, including epilepsy, pain, nausea and vomiting and others with reduced toxicity.

In one aspect, the invention provides stabilized solid nanoparticles comprising a cannabinoid and/or cannabinoid analog (exemplary structures illustrated in FIGS. 1-4) and at least one Ostwald ripening inhibitor. In some embodiments, the stabilized nanoparticles comprise albumin.

In another aspect, the invention provides a composition comprising a substantially stable and sterile filterable dispersion of solid nanoparticles in an aqueous medium,
wherein the solid nanoparticles comprise
   i) a cannabinoid and/or a cannabinoid analog; and
   ii) at least one Ostwald ripening inhibitor;
wherein the nanoparticles have a mean particle size of less than 220 nm as measured by photon correlation spectroscopy.

In some embodiments, the composition further comprises a biocompatible polymer as emulsifier. In some embodiments, the biocompatible polymer is human albumin or recombinant human albumin or PEG-human albumin or bovine serum albumin.

In some embodiments, the Ostwald ripening inhibitor is selected from the group
consisting of:
   (a) a mono-, di- or a tri-glyceride of a fatty acid;
   (b) a fatty acid mono- or di-ester of a C₂₋₁₀ diol;
   (c) a fatty acid ester of an alkanol or a cycloalkanoyl;
   (d) a wax;
   (e) a long chain aliphatic alcohol;
   (f) a hydrogenated vegetable oil;
   (g) cholesterol or fatty acid ester of cholesterol;
   (h) a ceramide;
   (i) a coenzyme Q10;
   (j) a lipoic acid or an ester of lipoic acid;
   (k) a phospholipid in an amount insufficient to form vesicles; and
   (l) combinations thereof.

In some embodiments, the cannabinoid or cannabinoid analog is selected from the group consisting of plant derived tetrahydrocannabidiol (THC), tetrahydrocannabivarin (THCV), synthetic tetrahydrocannabinol (THC or Dronabinol), delta 8 Tetrahydrocannabinol (A8-THC), plant derived cannabidiol (CBD), synthetic CBD, nabilone, HU-210, dexanabinol, Cannabicyclol (CBL), Cannabigerol (CBG) and Cannabichromene (CBC), Cannabielsoin (CBE) and Cannabinodiol (CBND), cannabinol (CBN), tetrahydrocannabinolic acid (THCA) and cannabidivarine (CBDV) and combinations thereof. In some embodiments, the cannabinoid is delta 8 Tetrahydrocannabinol (Δ8-THC).

In some embodiments, the Ostwald ripening inhibitor or mixture thereof, is sufficiently miscible with the cannabinoid or cannabinoid analog to form solid particles in the dispersion, wherein the particles comprise a substantially single-phase mixture of the cannabinoid or cannabinoid analog and the Ostwald ripening inhibitor or mixture thereof.

In some embodiments, the nanoparticles overcome significant obstacles in the way of developing oral treatments with these agents such as first pass hepatic metabolism, instability in the acidic gastric pH and/or low water solubility, leading to incomplete absorption. The benefits of nanoparticles for oral drug delivery include increased bioavailability, a higher rate of absorption, reduced fed/fasted variable absorption, improved dose proportionality, reduction of dosing frequency, and avoidance of uncontrolled precipitation after dosing (Natascia Bruni, et al., 2018).

In some embodiments, the nanoparticle drug delivery platform of the present invention allows development of drug products for pulmonary and nasal deliveries. The benefits are precision delivery to the target site, increased the uniformity of surface coverage, shorter nebulization times, reduced systemic toxicity, and accumulation of higher drug concentration at the target site. Therapeutic quantities of the drug can be delivered rapidly using ultrasonic nebulizers. Also, a much greater portion of the emitted dose can be deposited in the lung.

In some embodiments, the nanoparticle drug delivery platform also allows development of drug products for parenteral delivery. The benefits are high drug loading in aqueous formulations, avoidance of harsh vehicles (e.g., co-solvents, Solubilizer, pH extremes), readily syringable formulations facilitate use of traditional small-bore needles, and safety established for IV, IM and SC routes of administration.

The inventors have now surprisingly found that substantially stable dispersions of solid particles of diverse pharmaceutically active water insoluble cannabinoids and cannabinoid analogs in an aqueous medium can be also prepared using an oil-in-water emulsion process using protein or another polymer as a surfactant. The dispersions prepared according to the present invention exhibit little or no particle growth after the formation mediated by Ostwald ripening.

According to one aspect of the present invention there is provided a process for the preparation of a substantially stable dispersion of solid particles in an aqueous medium comprising:
combining (a) a first solution comprising a substantially water-insoluble substance, a water-immiscible organic solvent, optionally a water-miscible organic solvent and an Ostwald ripening inhibitor with (b) an aqueous phase comprising water and an emulsifier, preferabley a protein; forming an oil-in-water emulsion under high pressure homogenization and rapidly evaporating the water immiscible/miscible solvents under vacuum thereby producing solid particles comprising the Ostwald ripening inhibitor and the substantially water-insoluble substance;
wherein:
(i) the Ostwald ripening inhibitor is a non-polymeric hydrophobic organic compound that is substantially insoluble in water;
(ii) the Ostwald ripening inhibitor is less soluble in water than the substantially water-insoluble substance; and
(iii) the Ostwald ripening inhibitor is a phospholipid in an amount insufficient to form vesicles.

The process according to the present invention enables substantially stable dispersions of very small particles, especially nano-particles, to be prepared in high concentration without the particle growth.

The dispersion according to the present invention is substantially stable, by which we mean that the solid particles in the dispersion exhibit reduced or substantially no particle growth mediated by Ostwald ripening. By the term “reduced particle growth” it is meant that the rate of particle growth mediated by Ostwald ripening is reduced compared to particles prepared without the use of an Ostwald ripening inhibitor. By the term “substantialy no particle growth” it is meant that the mean particle size of the particles in the aqueous medium does not increase by more than 10% (more preferably by not more than 5%) over a period of 12-120 hours at 20° C. after the dipersion into the aqueous phase in the present process. By the term “substantially stable particle or nano-particle” it is meant that the mean particle size of the particles in the aqueous medium does not increase by more than 10% (more preferably by not more than 5%) over a period of 12-120 hours at 20° C. Preferably the particles exhibit substantially no particle growth over a period of 12-120 hours, more preferably over a period 24-120 hours and more preferably 48-120 hours.

It is to be understood that in those cases where the solid particles are prepared in an amorphous form the resulting particles will, generally, and eventually revert to a thermodynamically more stable crystalline form upon storage as an aqueous dispersion. The time taken for such dispersions to re-crystallise is dependent upon the substance and may vary from a few hours to several days. Generally, such re-crystallisation will result in particle growth and the formation of large crystalline particles which are prone to sedimentation from the dispersion. It is to be understood that the present invention does not prevent conversion of amorphous particles in the suspension into a crystalline state. The presence of the Ostwald ripening inhibitor in the particles according to the present invention significantly reduces or eliminates particle growth mediated by Ostwald ripening, as hereinbefore described. The particles are therefore stable to Ostwald ripening and the term “stable” used herein is to be construed accordingly.

The solid particles in the dispersion preferably have a mean particle size of less than 10 μm, more preferably less than 5 μm, still more preferably less than 1 μm and especially less than 500 nm. It is especially preferred that the particles in the dispersion have a mean particle size of from 10 to 500 nm, more especially from 50 to 300 nm and still more especially from 50 to 200 nm. The mean size of the particles in the dispersion may be measured using conventional techniques, for example by dynamic light scattering to measure the intensity-averaged particle size. Generally, the solid particles in the dispersion prepared according to the present invention exhibit a narrow unimodal particle size distribution.

The solid particles may be crystalline, semi-crystalline or amorphous. In an embodiment, the solid particles comprise a pharmacologically active substance in a substantially amorphous form. This can be advantageous as many pharmacological compounds exhibit increased bioavailability in amorphous form compared to their crystalline or semi-crystalline forms. The precise form of the particles obtained will depend upon the conditions used during the evaporation step of the process. Generally, the present process results in rapid evaporation of the emulsion and the formation of substantially amorphous particles.

In some embodiments, the invention provides a method for producing solid nanoparticles with mean diameter size of less than 220 nm, more preferably with a mean diameter size of about 20-200 nm and most preferably with a mean diameter size of about 50-180 nm. These solid nanoparticle suspensions can be sterile filtered through a 0.22 m filter and lyophilized. The sterile suspensions can be lyophilized in the form of a cake in vials with or without cryoprotectants such as sucrose, mannitol, trehalose or the like. The lyophilized cake can be reconstituted to the original solid nanoparticle suspensions, without modifying the nanoparticle size, stability or the drug potency, and the cake is stable for more than 24 months.

In another embodiment, the sterile-filtered solid nanoparticles can be lyophilized in the form of a cake in vials using cryoprotectants such as sucrose, mannitol, trehalose or the like. The lyophized cake can be reconstituted to the original nanoparticles, without modifying the particle size.

These nanoparticles can be administered by a variety of routes, preferably by parenteral, nasal, inhalation, and oral routes.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Chemical Structures of Tetrahydrocannabinol (THC), Cannabinol (CBN), Cannabidiol (CBD), Cannabicyclol (CBL), Cannabigerol (CBG) and Cannabichromene (CBC), Cannabielsoin (CBE) and Cannbinodiol (CBND).

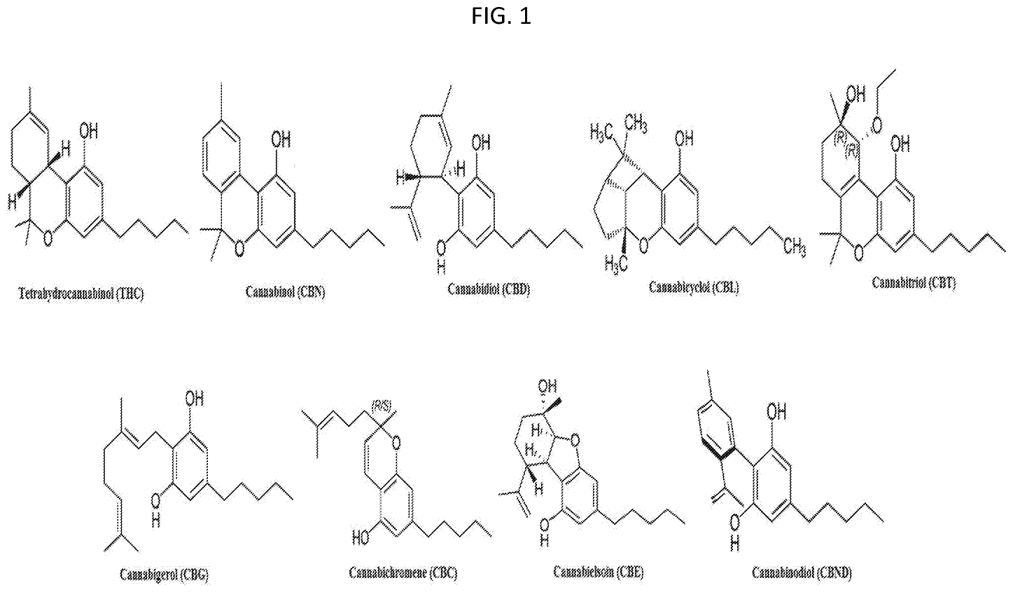

FIG. 2. Chemical Structure of Nabilone.

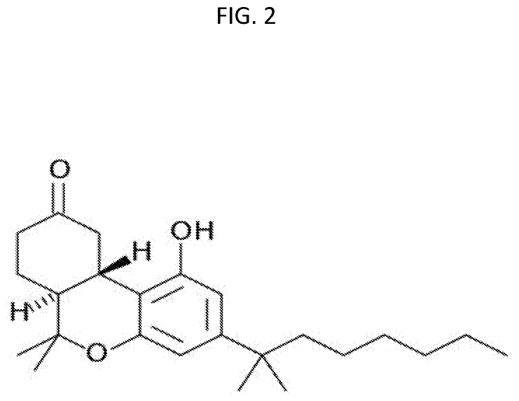

FIG. 3. Chemical Structure of HU-210 (1,1-Dimethylheptyl-11-Hydroxy-tetrahydrocannabinol).

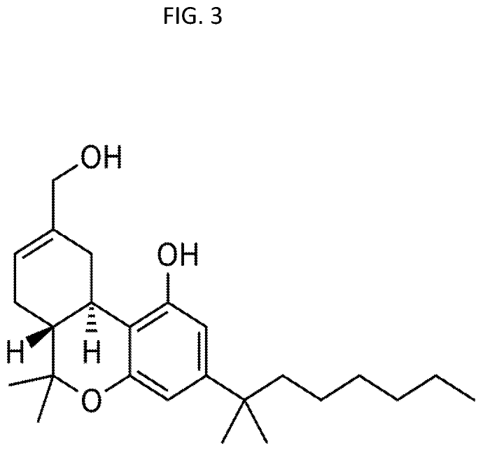

FIG. 4. Chemical Structure of Dexanabinol.

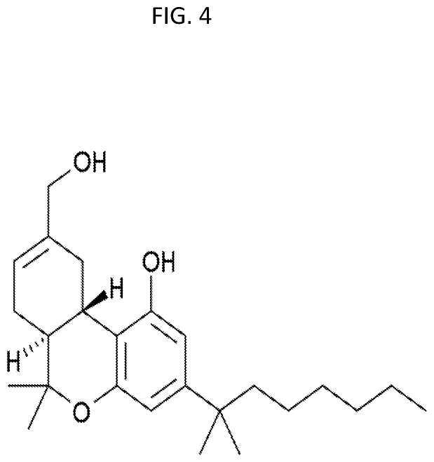

FIG. 5. The Particle Size Analysis of 4% Albumin after Homogenization with Chloroform and Ethanol (Measured Using Malvern Nano S).

FIG. 6. The Particle Size Distribution of Reconstituted Suspension stored at room temperature for 102 days in EXAMPLE 3 (Measured Using Malvern Zetasizer Nano S).

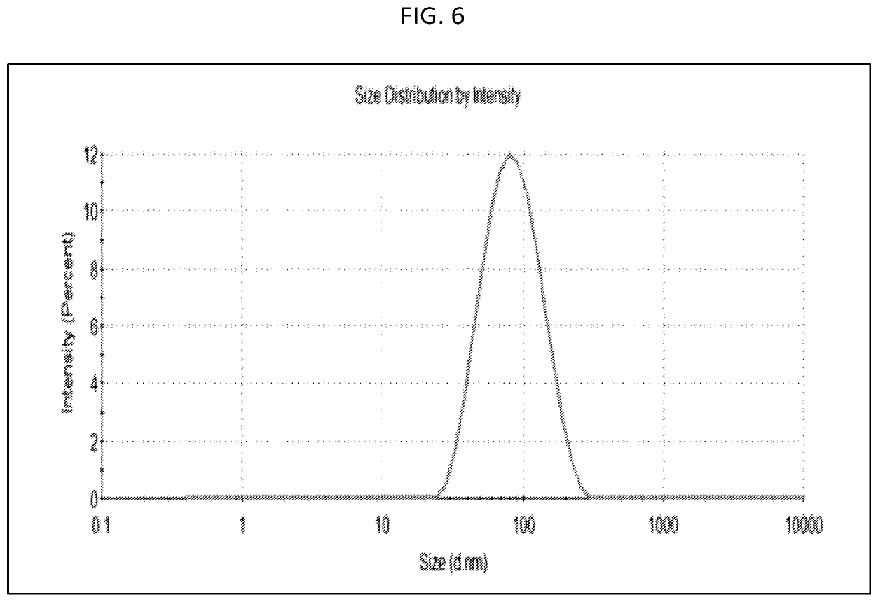

### DETAILED DESCRIPTION OF THE INVENTION

Reference will now be made in detail to the presently preferred embodiments of the invention which, together with the drawings and the following examples, explain the principles of the invention. These embodiments describe in enough detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

It is understood as “cannabinoids and cannabinoid analogs” in which most THC effects are mediated through agonistic actions at cannabinoid receptors. The mode of action of cannabidiol is not fully understood and several mechanisms have been proposed: (1) CBD acts as antagonist at the central CB1 receptor and was able to inhibit several CB1 mediated THC effects. CBD considerably reduced the receptor activation of a potent classical CB1 receptor agonist. (2) CBD stimulates the vanilloid receptor type 1 (VR1) with a maximum effect similar in efficacy to that of capsaicin.

As used herein, the term “μm” or the term “micrometer or micron” refers to a unit of measure of one one-millionth of a meter.

As used herein, the term “nm” or the term “nanometer” refers to a unit of measure of one one-billionth of a meter.

As used herein, the term “μg” or the term “microgram” refers to a unit of measure of one one-millionth of a gram.

As used herein, the term “ng” or the term “nanogram” refers to a unit of measure of one one-billionth of a gram.

As used herein, the term “mL” refers to a unit of measure of one one-thousandth of a liter.

As used herein, the term “nmol” refers to a unit of measure of one one-thousandth of a mole per liter.

As used herein, the term “biocompatible” describes a substance that does not appreciably alter or affect in any adverse way, the biological system into which it is introduced.

As used herein, the term “substantially water insoluble pharmaceutical substance or agent” means biologically active chemical compounds which are poorly soluble or almost insoluble in water. Examples of such compounds are plant derived tetrahydrocannabidiol (THC), synthetic tetrahydrocannabinol (THC or Dronabinol), cannabidiol (CBD), nabilone, HU-210, dexanabinol, cannabinol (CBN), cannabigerol (CBG), tetrahydrocannabinolic acid (THCA), cannabidivarine (CBDV), tetrahydrocannabivarin (THCV), delta 8 tetrahydrocannabinol (Δ8-THC) and the like.

In some embodiments, the solubility is in a range of 0-100 μg/mL. In some embodiments, the solubility is in a range of 0-75 μg/mL, 0-50 μg/mL, 0-25 μg/mL, or 0-10 μg/mL. In some embodiments, the solubility is in a range of 10-100 μg/mL, 20-80 μg/mL, or 25-50 μg/mL.

By the term “reduced particle growth” is meant that the rate of particle growth mediated by Ostwald ripening is reduced compared to particles prepared without the use of an Ostwald ripening inhibitor.

By the term “substantialy no particle growth” is meant that the mean particle size of the particles in the aqueous medium does not increase by more than 10% (more preferably by not more than 5%) over a period of 12-120 hours at 20° C. after the dipersion into the aqueous phase in the present process.

By the term “substantially stable particle or nano-particle” is meant that the mean particle size of the particles in the aqueous medium does not increase by more than 10% (more preferably by not more than 5%) over a period of 12-120 hours at 20° C. Preferably the particles exhibit substantially no particle growth over a period of 12-120 hours, more preferably over a period 24-120 hours and more preferably 48-120 hours.

In some embodiments, the term “cannabinoids or cannabinoid analogs,” as used herein, refers to plant derived tetrahydrocannabidiol (THC), synthetic tetrahydrocannabinol (THC or Dronabinol), cannabidiol (CBD), nabilone, HU-210, dexanabinol, cannabinol (CBN), cannabigerol (CBG), tetrahydrocannabinolic acid (THCA), and cannabidivarine (CBDV).

The term “Inhibitor” refers in general to the organic substances which are added to the substantially water insoluble substance in order to reduce the instability of the solid nanoparticles dispersed in an aqueous medium due to Ostwald ripening.

The term “phospholipid in an amount insufficient to form vesicles” refers to the amount of phospholipid or mixture thereof added as Ostwald ripening inhibitor which does not induce the nanoparticles produced by the invention to transform into liposomes or vesicles. In some embodiments, the amount of phospholipid insufficient to form vesicles ranges from 0-10% (w/w).

In one embodiment, the present invention provides solid nanoparticle formulations without particle growth due to Ostwald ripening of substantially water insoluble pharmaceutical substances selected from cannabinoids and cannabinoid analogs and methods of preparing and employing such formulations.

In some embodiments, the formulations/compositions further comprise one or more terpenes, such as cannabinoid-based terpenes.

The advantages of these nanoparticle formulations are that a substantially water insoluble cannabinoids and cannabinoid analogs is co-precipitated with inhibitors of Ostwald ripening. These compositions have been observed to provide a very low toxicity form of the cannabinoids and cannabinoid analogs that can be delivered in the form of nanoparticles or suspensions by slow infusions or by bolus injection or by other parenteral or oral delivery routes. These nanoparticles have sizes below 400 nm, preferably below 200 nm, and more preferably below 140 nm having hydrophilic proteins adsorbed onto the surface of the nanoparticles. These nanoparticles can assume different morphology; they can exist as amorphous particles or as crystalline particles.

By “substantially insoluble” it is meant a substance that has a solubility in water at 25° C. of less than 0.5 mg/ml, preferably less than 0.1 mg/ml and especially less than 0.05 mg/ml.

In some embodiments, the greatest effect on particle growth inhibition is observed when the substance has a solubility in water at 25° C. of more than 0.2 μg/ml. In a preferred embodiment the substance has a solubility in the range of from 0.05 μg/ml to 0.5 mg/ml, for example from 0.05 μg/ml to 0.05 mg/ml.

The solubility of the substance in water may be measured using a conventional technique. For example, a saturated solution of the substance is prepared by adding an excess amount of the substance to water at 25° C. and allowing the solution to equilibrate for 48 hours. Excess solids are removed by centrifugation or filtration and the concentration of the substance in water is determined by a suitable analytical technique such as HPLC.

In some embodiments, the nanoparticle produced by the present invention are approximately 60-190 nm in diameters, they will have a reduced uptake by the reticulo-endothelial system (RES), and, consequently, show a longer circulation time, increased biological and chemical stability, and increased accumulation in tumor-sites. Most importantly, the nanoparticle formulations can produce a marked enhancement of anti-tumor activity in mice with substantial reduction in toxicity as the nanoparticles can alter the pharmacokinetics and biodistribution. This can reduce toxic side effects and increase efficacy of the therapy.

### Ostwald Ripening Inhibitor:

The Ostwald ripening inhibitor is a non-polymeric hydrophobic organic compound that is less soluble in water than the substantially water-insoluble substance present in the water immiscible organic phase. Suitable Ostwald ripening inhibitors have a water solubility at 25° C. of less than 0.1 mg/L, more preferably less than 0.01 mg/L. In an embodiment of the invention the Ostwald ripening inhibitor has a solubility in water at 25° C. of less than 0.05 μg/ml, for example from 0.1 ng/ml to 0.05 μg/ml.

In an embodiment of the invention the Ostwald ripening inhibitor has a molecular weight of less than 2000, such as less than 500, for example less than 400. In another embodiment of the invention the Ostwald ripening inhibitor has a molecular weight of less than 1000, for example less than 600. For example, the Ostwald ripening inhibitor may have a molecular weight in the range of from 200 to 2000, preferably a molecular weight in the range of from 400 to 1000, more preferably from 200 to 600.

Suitable Ostwald ripening inhibitors include an inhibitor selected from classes (i) to (xi) or a combination of two or more such inhibitors:
(i) a mono-, di- or (more preferably) a tri-glyceride of a fatty acid. Suitable fatty acids include medium chain fatty acids containing from 8 to 12, more preferably from 8 to 10 carbon atoms or long chain fatty acids containing more than 12 carbon atoms, for example from 14 to 20 carbon atoms, more preferably from 14 to 18 carbon atoms. The fatty acid may be saturated, unsaturated or a mixture of saturated and unsaturated acids. The fatty acid may optionally contain one or more hydroxyl groups, for example ricinoleic acid. The glyceride may be prepared by well known techniques, for example, esterifying glycerol with one or more long or medium chain fatty acids. In a preferred embodiment the Ostwald ripening inhibitor is a mixture of triglycerides obtainable by esterifying glycerol with a mixture of long or, preferably, medium chain fatty acids. Mixtures of fatty acids may be obtained by extraction from natural products, for example from a natural oil such as palm oil. Fatty acids extracted from palm oil contain approximately 50 to 80% by weight decanoic acid and from 20 to 50% by weight of octanoic acid. The use of a mixture of fatty acids to esterify glycerol gives a mixture of glycerides containing a mixture of different acyl chain lengths. Long and medium chain triglycerides are commercially available. For example, a medium chain triglyceride (MCT) containing acyl groups with 8 to 12, more preferably 8 to 10 carbon atoms are prepared by esterification of glycerol with fatty acids extracted from palm oil, giving a mixture of triglycerides containing acyl groups with 8 to 12, more preferably 8 to 10 carbon atoms. This MCT is commercially available as Miglyol 812N (Huls, Germany). Other commercially available MCT's include Miglyol 810 and Miglyol 818 (Huls, Germany). A further suitable medium chain triglyceride is trilaurine (glycerol trilaurate). Commercially available long chain trigylcerides include glyceryl tri-stearate, glyceryl tri-palmitate, soybean oil, sesame oil, sunflower oil, castor oil or rape-seed oil.

Mono and di-glycerides may be obtained by partial esterification of glycerol with a suitable fatty acid, or mixture of fatty acids. If necessary, the mono- and di-glycerides may be separated and purified using conventional techniques, for example by extraction from a reaction mixture following esterification. When a mono-glyceride is used it is preferably a long-chain mono glyceride, for example a mono glyceride formed by esterification of glycerol with a fatty acid containing 18 carbon atoms;
(ii) a fatty acid mono- or (preferably) di-ester of a C₂₋₁₀ diol. Preferably the diol is an aliphatic diol which may be saturated or unsaturated, for example a C₂₋₁₀-alkane diol which may be a straight chain or branched chain diol. More preferably the diol is a C₂₋₆-alkane diol which may be a straight chain or branched chain, for example ethylene glycol or propylene glycol. Suitable fatty acids include medium and long chain fatty acids described above in relation to the glycerides. Preferred esters are di-esters of propylene glycol with one or more fatty acids containing from 10 to 18 carbon atoms, for example Miglyol 840 (Huls, Germany);
(iii) a fatty acid ester of an alkanol or a cycloalkanol. Suitable alkanols include C₁₋₂₀-alkanols which may be straight chain or branched chain, for example ethanol, propanol, isopropanol, n-butanol, sec-butanol or tert-butanol. Suitable cycloalkanols include C₃₋₆-cycloalkanols, for example cyclohexanol. Suitable fatty acids include medium and long chain fatty acids described above in relation to the glycerides. Preferred esters are esters of a C₂₋₆-alkanol with one or more fatty acids containing from 8 to 10 carbon atoms, or more preferably 12 to 29 carbon atoms, which fatty acid may be saturated or unsaturated. Suitable esters include, for example dodecyl dodecanoate or ethyl oleate;
(iv) a wax. Suitable waxes include esters of a long chain fatty acid with an alcohol containing at least 12 carbon atoms. The alcohol may be an aliphatic alcohol, an aromatic alcohol, an alcohol containing aliphatic and aromatic groups or a mixture of two or more such alcohols. When the alcohol is an aliphatic alcohol it may be saturated or unsaturated. The aliphatic alcohol may be straight chain, branched chain or cyclic. Suitable aliphatic alcohols include those containing more than 12 carbon atoms, preferably more than 14 carbon atoms especially more than 18 carbon atoms, for example from 12 to 40, more preferably 14 to 36 and especially from 18 to 34 carbon atoms. Suitable long chain fatty acids include those described above in relation to the glycerides, preferably those containing more than 14 carbon atoms especially more than 18 carbon atoms, for example from 14 to 40, more preferably 14 to 36 and especially from 18 to 34 carbon atoms. The wax may be a natural wax, for example bees wax, a wax derived from plant material, or a synthetic wax prepared by esterification of a fatty acid and a long chain alcohol. Other suitable waxes include petroleum waxes such as a paraffin wax;
(v) a long chain aliphatic alcohol. Suitable alcohols include those with 6 or more carbon atoms, more preferably 8 or more carbon atoms, such as 12 or more carbon atoms, for example from 12 to 30, for example from 14 to 28 carbon atoms. It is especially preferred that the long chain aliphatic alcohol has from 10 to 28, more especially from 14 to 22 carbon atoms. The alcohol may be straight chain, branched chain, saturated or unsaturated. Examples of suitable long chain alcohols include, 1-hexadecanol, 1-octadecanol, or 1-heptadecanol; or
(vi) a hydrogenated vegetable oil, for example hydrogenated castor oil;
(vii) cholesterol and fatty acid esters of cholesterol;
(viii) ceramides;
(ix) coenzyme Q10;
(x) phospholipids in an amount insufficient to form vesicles; and
(xi) lipoic acid, its derivatives and their esters.

In one embodiment of the present invention the Ostwald ripening inhibitor is selected from a long chain triglyceride and a long chain aliphatic alcohol containing from 6 to 22, preferably from 10 to 20 carbon atoms. Preferred long chain triglycerides and long chain aliphatic alcohols are as defined above. In a preferred embodiment the Ostwald ripening inhibitor is selected from a long chain triglyceride containing acyl groups with from 12 to 18 carbon atoms or a mixture of such triglycerides and an ester aliphatic alcohol containing from 10 to 22 carbon atoms (preferably 1-hexadecanol) or a mixture thereof (for example hexadecyl hexadecanoate).

In some embodiments, the Ostwald ripening inhibitor is selected from hydrogenated soy phosphatidylcholine and soy lecithin.

In another embodiment of the present invention the Ostwald ripening inhibitor is selected from an ester of cholesterol and cholesterol. Preferred cholesteryl ester is cholesteryl palmitate or stearate.

When the substantially water-insoluble substance is a pharmacologically active compound the Ostwald ripening inhibitor is preferably a pharmaceutically inert material.

The Ostwald ripening inhibitor is present in the particles in a quantity sufficient to prevent Ostwald ripening of the particles in the suspension. Preferably the Ostwald ripening inhibitor will be the minor component in the solid particles formed in the present process comprising the Ostwald ripening inhibitor and the substantially water-insoluble substance. Preferably, therefore, the Ostwald ripening inhibitor is present in a quantity that is just sufficient to prevent Ostwald ripening of the particles in the dispersion, thereby minimizing the amount of Ostwald ripening inhibitor present in the particles.

In embodiments of the present invention the weight fraction of Ostwald ripening inhibitor relative to the total weight of Ostwald ripening inhibitor and substantially water-insoluble substance (i.e. weight of Ostwald ripening inhibitor/(weight of Ostwald ripening inhibitor+weight of substantially water-insoluble substance)) is from 0.01 to 0.99, preferably from 0.05 to 0.95, especially from 0.2 to 0.95 and more especially from 0.3 to 0.95. In a preferred embodiment the weight fraction of Ostwald ripening inhibitor relative to the total weight of Ostwald ripening inhibitor and substantially water-insoluble substance is less than 0.95, more preferably 0.9 or less, for example from 0.2 to 0.9, such as from 0.3 to 0.9, for example about 0.8. This is particularly preferred when the substantially water-insoluble substance is a pharmacologically active substance and the Ostwald ripening inhibitor is relatively non-toxic (e.g. a weight fraction above 0.8) which may not give rise to unwanted side effects and/or affect the dissolution rate/bioavailability of the pharmacologically active substance when administered in vivo.

In embodiments, the weight fraction of Ostwald ripening inhibitor relative to the total weight of Ostwald ripening inhibitor and substantially water-insoluble substance is between 0.40 to 0.65. In embodiments, the weight fraction of Ostwald ripening inhibitor relative to the total weight of Ostwald ripening inhibitor and substantially water-insoluble substance is between 0.4 to 0.6. In embodiments, the weight fraction of Ostwald ripening inhibitor relative to the total weight of Ostwald ripening inhibitor and substantially water-insoluble substance is between 0.45 to 0.60. In embodiments, the weight fraction of Ostwald ripening inhibitor relative to the total weight of Ostwald ripening inhibitor and substantially water-insoluble substance is between 0.45 to 0.55. In embodiments, the weight fraction of Ostwald ripening inhibitor relative to the total weight of Ostwald ripening inhibitor and substantially water-insoluble substance is about 0.5. Without being bound by theory, the release kinetics of the substantially water-insoluble substance can be manipulated by varying the weight fraction of Ostwald ripening inhibitor relative to the total weight of Ostwald ripening inhibitor and substantially water-insoluble substance. In some embodiments, the release kinetics can be enhanced when the weight fraction of Ostwald ripening inhibitor relative to the total weight of Ostwald ripening inhibitor and substantially water-insoluble substance is between from 0.40 to 0.65, more preferably about 0.5.

Furthermore, it has been found that in general a low weight ratio of Ostwald ripening inhibitor to the Ostwald ripening inhibitor and the substantially water-insoluble substance (i.e. less than 0.5) is sufficient to prevent particle growth by Ostwald ripening, thereby allowing small (preferably less than 1000 nm, preferably less than 500 nm) stable particles to be prepared. A small and constant particle size is often desirable, especially when the substantially water-insoluble substance is a pharmacologically active material that is used, for example, for intravenous administration.

One application of the dispersions prepared by the process according to the present invention is the study of the toxicology of a pharmacologically active compound. The dispersions prepared according to the present process can exhibit improved bioavailability compared to dispersions prepared using alternative processes, particularly when the particle size of the substance is less than 500 nm. In this application it is advantageous to minimize the amount of Ostwald ripening inhibitor relative to the active compound so that any effects on the toxicology associated with the presence of the Ostwald ripening inhibitor are minimized.

When the substantially water-insoluble substance has an appreciable solubility in the Ostwald ripening inhibitor the weight ratio of Ostwald ripening inhibitor to substantially water-insoluble substance should be selected to ensure that the amount of substantially water-insoluble substance exceeds that required to form a saturated solution of the substantially water-insoluble substance in the Ostwald ripening inhibitor. This ensures that solid particles of the substantially water-insoluble substance are formed in the dispersion. This is important when the Ostwald ripening inhibitor is a liquid at the temperature at which the dispersion is prepared (for example ambient temperature) to ensure that the process does not result in the formation liquid droplets comprising a solution of the substantially water-insoluble substance in the Ostwald ripening inhibitor, or a two phase system comprising the solid substance and large regions of the liquid Ostwald ripening inhibitor.

Without wishing to be bound by theory the inventors believe that systems in which there is a phase separation between the substance and Ostwald ripening inhibitor in the particles are more prone to Ostwald ripening than those in which the solid particles form a substantially single-phase system. Accordingly, in a preferred embodiment the Ostwald ripening inhibitor is sufficiently miscible in the substantially water-insoluble material to form solid particles in the dispersion comprising a substantially single-phase mixture of the substance and the Ostwald ripening inhibitor. The composition of the particles formed according to the present invention may be analyzed using conventional techniques, for example analysis of the (thermodynamic) solubility of the substantially water-insoluble substance in the Ostwald ripening inhibitor, melting entropy and melting points obtained using routine differential scanning calorimetry (DSC) techniques to thereby detect phase separation in the solid particles. Furthermore, studies of nano-suspensions using nuclear magnetic resonance (NMR) (e.g. line broadening of either component in the particles) may be used to detect phase separation in the particles.

Generally, the Ostwald ripening inhibitor should have a sufficient miscibility with the substance to form a substantially single-phase particle, by which is meant that the Ostwald ripening inhibitor is molecularly dispersed in the solid particle or is present in small domains of Ostwald ripening inhibitor dispersed throughout the solid particle. It is thought that for many substances the substance/Ostwald ripening inhibitor mixture is a non-ideal mixture by which it is meant that the mixing of two components is accompanied by a non-zero enthalpy change.

It should be noted that apart from stabilizing the nanoparticles, the Oswald ripening inhibitors can improve the therapeutic efficacy and toxicity of the substantially insoluble substance when administered to mammals. Thus, the Ostwald ripening inhibitors can have multiple physiological effects apart from stabilizing the nanoparticles.

Preparation of the Inventive Nanoparticles: In some embodiments, in order to form the solid nanoparticles dispersed in an aqueous medium, a substantially water insoluble pharmaceutical substance and the Ostwald ripening inhibitor(s) are dissolved in a suitable solvent (e.g., chloroform, methylene chloride, ethyl acetate, ethanol, tetrahydrofuran, dioxane, acetonitrile, acetone, dimethyl sulfoxide, dimethyl formamide, methyl pyrrolidinone, or the like, as well as mixtures of any two or more thereof). Additional solvents contemplated for use in the practice of the present invention include soybean oil, coconut oil, olive oil, safflower oil, cotton seed oil, sesame oil, orange oil, limonene oil, C₁-C₂₀ alcohols, C₂-C₂₀ esters, C₃-C₂₀ ketones, polyethylene glycols, aliphatic hydrocarbons, aromatic hydrocarbons, halogenated hydrocarbons and combinations thereof.

In some embodiments, in the next stage, in order to make the solid nanoparticles, a protein (e.g., human serum albumin) is added (into the aqueous phase) to act as a stabilizing agent or an emulsifier for the formation of stable nanodroplets. Protein is added at a concentration in the range of about 0.05 to 25% (w/v), more preferably in the range of about 0.5%-10% (w/v).

In some embodiments, in the next stage, in order to make the solid nanoparticles, an emulsion is formed by homogenization under high pressure and high shear forces. Such homogenization is conveniently carried out in a high-pressure homogenizer, typically operated at pressures in the range of about 3,000 up to 30,000 psi. Preferably, such processes are carried out at pressures in the range of about 6,000 up to 25,000 psi. The resulting emulsion comprises very small nanodroplets of the nonaqueous solvent containing the substantially water insoluble pharmaceutical substance, the Ostwald ripening inhibitor and other agents. Acceptable methods of homogenization include processes imparting high shear and cavitation such as high-pressure homogenization, high shear mixers, sonication, high shear impellers, and the like.

In some embodiments, in order to make the solid nanoparticles, the solvent is evaporated under reduced pressure to yield a colloidal system composed of solid nanoparticles of a substantially water insoluble cannabinoids and cannabinoid analogs and the Ostwald ripening inhibitor(s) in solid form and protein. Acceptable methods of evaporation include the use of rotary evaporators, falling film evaporators, spray driers, freeze driers, and the like. Following evaporation of solvent, the liquid suspension may be dried to obtain a powder containing the pharmacologically active agent and protein. The resulting powder can be redispersed at any convenient time into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals. Methods contemplated for obtaining this powder include freeze-drying, spray drying, and the like.

The solid nanoparticles herein can be administered for pharmaceutical or for recpreational use. In some embodiments, the solid nanoparticles can be formulated for ingestion, e.g., capsules, tablets, oral drops, lozenges, lollipops, and food preparations, i.e., “edibles” (aka ingestible, in contrast to sublingual or buccal absorption), such as baked goods including brownies, muffins, cookies, etc., chocolates, chews, beverages, fruit snacks, gum drops, soft candies (such as gummies), hard candies, liquid shots, and the like. In some embodiments, they can be formulated for inhalation into the lungs (e.g., by a heat vaporizer (“vape”) or nebulizer).

In some embodiments, the solid nanoparticles as described herein are added to compositions in the manufacture of food products. In some embodiments, a powder composition of solid nanoparticles is added to a food ingredient composition or product during the food manufacture process. In some embodiments, a suspension of solid nanoparticles is added to a food ingredient composition or product during the food manufacture process. In some embodiments, a suspension comprising the solid nanoparticles as described herein is combined with a gelatin composition in order to make a gelatin based food product, such as “gummies,” for example, as described in the below Examples. In some embodiments, a powder composition of solid nanoparticles is added to a gelatin composition to make a gummy based product.

In some embodiments, the food product comprises a composition of solid nanoparticles comprising Δ8-THC. In some embodiments, the food product is a gummy, gelatin based soft candy.

In accordance with a specific embodiment of the present invention, there is provided a method for the formation of unusually small submicron solid particles containing a substantially water insoluble cannabinoids and cannabinoid analogs and an Ostwald ripening inhibitor, i.e., particles which are less than 200 nanometers in diameter. Such particles are capable of being sterile-filtered before use in the form of a liquid suspension. The ability to sterile-filter the end product of the invention formulation process (i.e., the substantially water insoluble cannabinoid and cannabinoid analog nanoparticles) is of great importance since it is impossible to sterilize dispersions which contain high concentrations of protein (e.g., serum albumin) by conventional means such as autoclaving.

In some embodiments, in order to obtain sterile-filterable solid nanoparticles of substantially water insoluble cannabinoids and cannabinoid analogs (i.e., particles <200 nm), the substantially water insoluble cannabinoids and cannabinoid analogs and the Ostwald ripening inhibitor(s) are initially dissolved in a substantially water immiscible organic solvent (e.g., a solvent having less than about 5% solubility in water, such as, for example, chloroform) at high concentration, thereby forming an oil phase containing the substantially water insoluble cannabinoids and cannabinoid analogs, the Ostwald ripening inhibitor and other agents. Suitable solvents are set forth above. Next, a water miscible organic solvent (e.g., a solvent having greater than about 10% solubility in water, such as, for example, ethanol) is added to the oil phase at a final concentration in the range of about 1%-99% v/v, more preferably in the range of about 5%-25% v/v of the total organic phase. The water miscible organic solvent can be selected from such solvents as ethyl acetate, ethanol, tetrahydrofuran, dioxane, acetonitrile, acetone, dimethyl sulfoxide, dimethyl formamide, methyl pyrrolidinone, and the like. Alternatively, the mixture of water immiscible solvent with the water miscible solvent is prepared first, followed by dissolution of the substantially water insoluble cannabinoids and cannabinoid analogs, the Ostwald ripening inhibitor and other agents in the mixture. It is believed that the water miscible solvent in the organic phase acts as a lubricant on the interface between the organic and aqueous phases resulting in the formation of fine oil in water emulsion during homogenization.

In some embodiments, in the next stage, for the formation of solid nanoparticles of substantially water insoluble pharmaceutical substances with reduced Ostwald growth, human serum albumin or any other suitable stabilizing agent as described above, is dissolved in aqueous media. This component acts as an emulsifying agent for the formation of stable nanodroplets. Optionally, a sufficient amount of the first organic solvent (e.g. chloroform) is dissolved in the aqueous phase to bring it close to the saturation concentration. A separate, measured amount of the organic phase (which now contains the substantially water insoluble cannabinoids and cannabinoid analogs, the first organic solvent and the second organic solvent) is added to the saturated aqueous phase, so that the phase fraction of the organic phase is between about 0.5%-15% v/v, and more preferably between 1% and 8% v/v. Next, a mixture composed of micro and nanodroplets is formed by homogenization at low shear forces. This can be accomplished in a variety of ways, as can readily be identified by those of skill in the art, employing, for example, a conventional laboratory homogenizer operated in the range of about 2,000 up to about 15,000 rpm. This is followed by homogenization under high pressure (i.e., in the range of about 3,000 up to 30,000 psi). The resulting mixture comprises an aqueous protein solution (e.g., human serum albumin), the substantially water insoluble cannabinoids and cannabinoid analogs, Ostwald ripening inhibitor(s), other agents, the first solvent and the second solvent. Finally, solvent is rapidly evaporated under vacuum to yield a colloidal dispersion system (solids of a substantially water insoluble cannabinoids and cannabinoid analogs, the Ostwald ripening inhibitor and other agents and protein) in the form of extremely small nanoparticles (i.e., particles in the range of about 50 nm-200 nm diameter), and thus can be sterile-filtered. The preferred size range of the particles is between about 50 nm-170 nm, depending on the formulation and operational parameters.

In some embodiments, the solid nanoparticles prepared in accordance with the present invention may be further converted into powder form by removal of the water there from, e.g., by lyophilization at a suitable temperature-time profile. The protein (e.g., human serum albumin) itself acts as a cryoprotectant, and the powder is easily reconstituted by addition of water, saline or buffer, without the need to use such conventional cryoprotectants as mannitol, sucrose, trehalose, glycine, and the like. While not required, it is of course understood that conventional cryoprotectants may be added to invention formulations if so desired. The solid nanoparticles containing substantially water insoluble cannabinoids and cannabinoid analogs allows for the delivery of high doses of the cannabinoids and cannabinoid analogs in relatively small volumes.

According to this embodiment of the present invention, the solid nanoparticles containing substantially water insoluble cannabinoids and cannabinoid analogs has a cross-sectional diameter of no greater than about 2 microns. A cross-sectional diameter of less than 1 microns is more preferred, while a cross-sectional diameter of less than 0.22 micron is presently the most preferred for the intravenous route of administration.

Proteins contemplated for use as stabilizing agents in accordance with the present invention include albumins (which can contain 35 cysteine residues), immunoglobulins, caseins, insulins (which contain 6 cysteines), hemoglobins (which contain 6 cysteine residues per α2 β2 unit), lysozymes (which contain 8 cysteine residues), immunoglobulins, α-2-macroglobulin, fibronectins, vitronectins, fibrinogens, lipases, and the like. Proteins, peptides, enzymes, antibodies and combinations thereof, are general classes of stabilizers contemplated for use in the present invention.

In some embodiments, the protein is albumin or a fragment thereof. In some embodiments, the protein is human serum albumin or a fragment thereof. In some embodiments, the protein is bovine serum albumin or a fragment thereof. In some embodiments, the protein is alpha-lactalbumin.

In some embodiments, the protein is water soluble soy protein(s) (“Aquafaba).

In one embodiment, a protein for use is albumin. Human serum albumin (HSA) is the most abundant plasma protein (˜640 μM) and is non-immunogenic to humans. The protein is principally characterized by its remarkable ability to bind a broad range of hydrophobic, small molecule ligands including fatty acids, bilirubin, thyroxine, bile acids and steroids; it serves as a solubilizer and transporter for these compounds and, in some cases, provides important buffering of the free concentration. HSA also binds a wide variety of drugs in two primary sites which overlap with the binding locations of endogenous ligands. The protein is a helical monomer of 66 kD containing three homologous domains (I-III) each of which is composed of A and B subdomains. The measurements on 44rythrosin-bovine serum albumin complex in neutral solution, using the phosphorescence depolarization techniques, are consistent with the absence of independent motions of large protein segments in solution of BSA, in the time range from nanoseconds to fractions of milliseconds. These measurements support a heart shaped structure (8 nm×8 nm×3.2 nm) of albumin in neutral solution of BSA as in the crystal structure of human serum albumin. Another advantage of albumin is its ability to transport drugs into tumor sites. Specific antibodies may also be utilized to target the nanoparticles to specific locations. HSA contains only one free sulfhydryl group as the residue Cys34 and all other Cys residues are bridged with disulfide bonds (Sugio S, et al., 1999).

In the preparation of the inventive compositions, a wide variety of organic media can be employed to suspend or dissolve the substantially water insoluble cannabinoids and cannabinoid analogs. Organic media contemplated for use in the practice of the present invention include any nonaqueous liquid that is capable of suspending or dissolving the cannabinoids and cannabinoid analogs but does not chemically react with either the polymer employed as emulsifier, or the pharmacologically active agent itself. Examples include vegetable oils (e.g., soybean oil, olive oil, and the like), coconut oil, safflower oil, cotton seed oil, sesame oil, orange oil, limonene oil, aliphatic, cycloaliphatic, or aromatic hydrocarbons having 4-30 carbon atoms (e.g., n-dodecane, n-decane, n-hexane, cyclohexane, toluene, benzene, and the like), aliphatic or aromatic alcohols having 2-30 carbon atoms (e.g., octanol, and the like), aliphatic or aromatic esters having 2-30 carbon atoms (e.g., ethyl caprylate (octanoate), and the like), alkyl, aryl, or cyclic ethers having 2-30 carbon atoms (e.g., diethyl ether, tetrahydrofuran, and the like), alkyl or aryl halides having 1-30 carbon atoms (and optionally more than one halogen substituent, e.g., CH₃Cl, CH₂Cl₂, CH₂Cl—CH₂Cl, and the like), ketones having 3-30 carbon atoms (e.g., acetone, methyl ethyl ketone, and the like), polyalkylene glycols (e.g., polyethylene glycol, and the like), or combinations of any two or more thereof.

Especially preferred combinations of organic media contemplated for use in the practice of the present invention typically have a boiling point of no greater than about 200° C., and include volatile liquids such as dichloromethane, chloroform, ethyl acetate, benzene, and the like (i.e., solvents that have a high degree of solubility for the cannabinoids and cannabinoid analogs, and are soluble in the other organic medium employed), along with a higher molecular weight (less volatile) organic medium. When added to the other organic medium, these volatile additives help to drive the solubility of the cannabinoids and cannabinoid analogs into the organic medium. This is desirable since this step is usually time consuming. Following dissolution, the volatile component may be removed by evaporation (optionally under vacuum).

In some embodiments, the solid nanoparticle formulations prepared in accordance with the present invention may further contain a certain quantity of biocompatible surfactants to further stabilize the emulsion during the homogenization in order to reduce the droplet sizes. These biocompatible surfactants can be selected from natural lecithins such as egg lecithin, soy lecithin; plant monogalactosyl diglyceride (hydrogenated) or plant digalactosyl diglyceride (hydrogenated); synthetic lecithins such as dihexanoyl-L-α-lecithin, dioctanoyl-L-α.-lecithin, didecanoyl-L-α.-lecithin, didodecanoyl-L-α-lecithin, ditetradecanoyl-L-α-lecithin, dihexadecanoyl-L-α-lecithin, dioctadecanoyl-L-α-lecithin, dioleoyl-L-α-lecithin, dilinoleoyl-L-α-lecithin, α-palmito, β-oleoyl-L-α-lecithin, L-α-glycerophosphoryl choline; polyoxyethylated hydrocarbons or vegetable oils such as Cremaphor® EL or RH40, Emulphor® EL-620P or EL-719, Arlacel®-186, Pluronic® F-68; sorbitan esters such as sorbitan monolaurate, sorbitan monostearate, sorbitan monopalmitate, sorbitan tristearate, sorbitan monooleate; PEG fatty acid esters such as PEG 200 dicocoate, PEG 300 distearate, PEG 400 sesquioleate, PEG 400 dioleate; ethoxylated glycerine esters such as POE(20) glycerol monostearate, POE(20) glycerol monooleate; ethoxylated fatty amines such as POE(15) cocorylamine, POE(25) cocorylamine, POE(80) oleylamine; ethoxylated sorbitan esters such as POE(20) sorbitan Monolaurate, POE(20) sorbitan monostearate, POE(20) sorbitan tristearate, POE(20) sorbitan trioleate; ethoxylated fatty acids such as POE(5) oleic acid, POE(5) coconut fatty acid, POE(14) coconut fatty acid, POE(9) stearic acid, POE(40) stearic acid; alcohol-fatty acid esters such as 2-ethylhexyl palmitate, isobutyl oleate, di-tridecyl adipate; ethoxylated alcohols such as POE(2)-2-ethyl hexyl alcohol, POE(10) cetyl alcohol, POE(4) decyl alcohol, POE(6) lauryl alcohol; alkoxylated castor oils such as POE(5) castor oil, POE(25) castor oil, POE(25) hydrogenated castor oil; glycerine esters such as glycerol monostearate, glyceryl behenate, glycerol tri caprylate; polyethylene glycols such as polyethylene glycol-200, polyethylene glycol-300, polyethylene glycol-400, polyethylene glycol 600, polyethylene glycol 1000; sugar esters such as sucrose fatty acid esters. The percentage of the biocompatible surfactants in the formulation can vary from 0.002% to 1% by weight.

In some embodiments, the solid nanoparticle formulations prepared in accordance with the present invention may further contain a polymer such as, but not limited to, lactic acid-based polymers such as polylactides e.g. poly(D,L-lactide) i.e. PLA; glycolic acid-based polymers such as polyglycolides (PGA) e.g. Lactel® from Durect; poly(D,L-lactide-co-glycolide) i.e. PLGA, (Resomer® RG-504, Resomer® RG-502, Resomer® RG-504H, Resomer® RG-502H, Resomer® RG-504S, Resomer® RG-502S, from Boehringer, Lactel® from Durect); polycaprolactones such as Poly(e-caprolactone) i.e. PCL (Lactel® from Durect); polyanhydrides; poly(sebacic acid) SA; poly(ricenolic acid) RA; poly(fumaric acid), FA; poly(fatty acid dimer), FAD; poly(terephthalic acid), TA; poly(isophthalic acid), IPA; poly(p-{carboxyphenoxy}methane), CPM; poly(p-{carboxyphenoxy}propane), CPP; poly(p-{carboxyphenoxy}hexane)s CPH; polyamines, polyurethanes, polyesteramides, polyorthoesters {CHDM: cis/trans-cyclohexyl dimethanol, HD:1,6-hexanediol. DETOU: (3,9-diethylidene-2,4,8,10-tetraoxaspiro undecane)}; polydioxanones; polyhydroxybutyrates; polyalkylene oxalates; polyamides; polyesteramides; polyurethanes; polyacetals; polyketals; polycarbonates; polyorthocarbonates; polysiloxanes; polyphosphazenes; succinates; hyaluronic acid: poly(malic acid); poly(amino acids); polyhydroxyvalerates; polyalkylene succinates; polyvinylpyrrolidone; polystyrene; synthetic cellulose esters; polyacrylic acids; polybutyric acid; triblock copolymers (PLGA-PEG-PLGA), triblock copolymers (PEG-PLGA-PEG), poly(N-isopropylacrylamide) (PNIPAAm), poly(ethylene oxide)-poly(propylene oxide)-poly(ethylene oxide) tri-block copolymers (PEO-PPO-PEO), poly valeric acid; polyethylene glycol; polyhydroxyalkylcellulose; chitin; chitosan; polyorthoesters and copolymers, terpolymers; poly(glutamic acid-co-ethyl glutamate) and the like, or mixtures thereof.

In some embodiments, the solid nanoparticle formulations prepared in accordance with the present invention may further contain certain chelating agents. The biocompatible chelating agent to be added to the formulation can be selected from ethylenediaminetetraacetic acid (EDTA), diethylenetriaminepentaacetic acid (DTPA), ethylene glycol-bis(β-aminoethyl ether)-tetraacetic acid (EGTA), N-(hydroxyethyl)-ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.

In some embodiments, the nanoparticle formulations prepared in accordance with the present invention may further contain certain antioxidants which can be selected from ascorbic acid derivatives such as ascorbic acid, erythorbic acid, sodium ascorbate, ascorbyl palmitate, retinyl palmitate; thiol derivatives such as thioglycerol, cysteine, acetylcysteine, cystine, dithioerythreitol, dithiothreitol, gluthathione; tocopherols; propyl gallate; butylated hydroxyanisole; butylated hydroxytoluene; sulfurous acid salts such as sodium sulfate, sodium bisulfite, acetone sodium bisulfite, sodium metabisulfite, sodium sulfite.

In some embodiments, the nanoparticle formulations prepared in accordance with the present invention may further contain certain preservatives if desired. The preservative for adding into the present inventive formulation can be selected from phenol, chlorobutanol, benzylalcohol, benzoic acid, sodium benzoate, methylparaben, propylparaben, benzalkonium chloride and cetylpyridinium chloride.

In some embodiments, the solid nanoparticles containing a substantially water insoluble pharmaceutical substance and the Ostwald ripening inhibitor with protein, prepared as described above, are delivered as a suspension in a biocompatible aqueous liquid. This liquid may be selected from water, saline, a solution containing appropriate buffers, a solution containing nutritional agents such as amino acids, sugars, proteins, carbohydrates, vitamins or fat, and the like.

In some embodiments, for increasing the long-term storage stability, the solid nanoparticle formulations may be frozen and lyophilized in the presence of one or more protective agents such as sucrose, mannitol, trehalose or the like. Upon rehydration of the lyophilized solid nanoparticle formulations, the suspension retains essentially all the substantially water insoluble cannabinoids and cannabinoid analogs previously loaded and the particle size. The rehydration is accomplished by simply adding purified or sterile water or 0.9% sodium chloride injection or 5% dextrose solution followed by gentle swirling of the suspension. The potency of the substantially water insoluble cannabinoids and cannabinoid analogs in a solid nanoparticle formulation is not lost after lyophilization and reconstitution.

In some embodiments, the solid nanoparticle formulation of the present invention is shown to be less prone to Ostwald ripening due to the presence of the Ostwald ripening inhibitors and are more stable in solution than the formulations disclosed in the prior art.

For the treatment of subjects, e.g., human patients, the subject can be administered or provided a pharmaceutical composition of the invention. The composition can be administered to the patient in therapeutically effective amounts. The pharmaceutical composition can be administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. The pharmaceutical composition may be administered parenterally, when possible, at the target site, or intravenously. Therapeutic compositions of the invention can be administered to a patient or subject systemically, parenterally, or locally.

The dose and dosage regimen depend upon a variety of factors readily determined by a physician, such as the nature of the disease or condition to be treated, the patient, and the patient's history. Generally, a therapeutically effective amount of a pharmaceutical composition is administered to a patient. In particular embodiments, the amount of active compound administered is in the range of about 0.01 mg/kg to about 20 mg/kg of patient body weight. The administration can comprise one or more separate administrations, or by continuous infusion. The progress therapy can be readily monitored by conventional methods and assays and based on criteria known to the physician or other persons of skill in the art.

In another embodiment, the invention provides a method of treating a disease or condition in a subject, comprising administering to the subject an effective amount of the pharmaceutical composition of the invention as described herein.

As used herein, “treat” and all its forms and tenses (including, for example, treating, treated, and treatment) refers to therapeutic and prophylactic treatment. In certain aspects of the invention, those in need of treatment include those already with a pathological disease or condition of the invention (including, for example, a cancer), in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of skill in the art realizes that a treatment may improve the patient's condition but may not be a complete cure of the disease or pathological condition.

A “therapeutically effective amount” or “effective amount” can be administered to the subject. As used herein a “therapeutically effective amount” or “effective amount” is an amount sufficient to decrease, suppress, or ameliorate one or more symptoms associated with the disease or condition.

The subject to be treated herein is not limiting. In some embodiments, the subject to be treated is a mammal, bird, reptile or fish. Mammals that can be treated in accordance with the invention, include, but are not limited to, humans, dogs, cats, horses, mice, rats, guinea pigs, sheep, cows, pigs, monkeys, apes and the like. The term “patient” and “subject” are used interchangeably. In some embodiments, the subject is a human.

The therapeutic composition can be administered one time or more than one time, for example, more than once per day, daily, weekly, monthly, or annually. The duration of treatment is not limiting. The duration of administration of the therapeutic agent can vary for each individual to be treated/administered depending on the individual cases and the diseases or conditions to be treated. In some embodiments, the therapeutic agent can be administered continuously for a period of several days, weeks, months, or years of treatment or can be intermittently administered where the individual is administered the therapeutic agent for a period of time, followed by a period of time where they are not treated, and then a period of time where treatment resumes as needed to treat the disease or condition. For example, in some embodiments, the individual to be treated is administered the therapeutic agent of the invention daily, every other day, every three days, every four days, 2 days per week 3 days per week, 4 days per week, 5 days per week or 7 days per week. In some embodiments, the individual is administered the therapeutic agent for 1 week, 2 weeks, 3 weeks, 4 weeks, 1 month, 2 months, 3 months, 4 months, 5 months, 6 months, 7 months, 8 months, 9 months, 10 months, 11 months, 1 year or longer.

In some embodiments, the disease or condition to be treated include epilepsy/seizure, pain, Alzheimer's, anorexia, anxiety, atherosclerosis, arthritis cancer, colitis/Crohn's, depression, diabetes, fibromyalgia, glaucoma, irritable bowel, multiple sclerosis, neurodegeneration, obesity, osteoporosis, Parkinson's, PTSD, schizophrenia, substance dependence/addiction, and stroke/traumatic brain injury.

In some embodiments, the subject is administered one or more additional therapeutic agents. In some embodiments, the one or more additional therapeutic agents are those commonly used to treat cancer.

The examples provided here are not intended, however, to limit or restrict the scope of the present invention in any way and should not be construed as providing conditions, parameters, reagents, or starting materials which must be utilized exclusively in order to practice the art of the present invention.

### EXAMPLES

### Example 1. Effect of Emulsification on Human Serum Albumin

An organic phase was prepared by mixing 3.5 mL of chloroform and 0.6 mL of dehydrated ethanol. A 4% human albumin solution was prepared by dissolving 2 gm of human albumin (Sigma-Aldrich Co, USA) in 50 mL of sterile Type I water. The pH of the human albumin solution was adjusted to 6.0-6.7 by adding either 1N hydrochloric acid or 1N sodium hydroxide solution in sterile water. The above organic solution was added to the albumin phase and the mixture was pre-homogenized with an IKA homogenizer at 6000-10000 RPM (IKA Works, Germany). The resulting emulsion was subjected to high-pressure homogenization (Avestin Inc, USA). The pressure was varied between 20,000 and 30,000 psi and the emulsification process was continued for 5-8 passes. During homogenization the emulsion was cooled between 5° C. and 10° C. by circulating the coolant through the homogenizer from a temperature-controlled heat exchanger (Julabo, USA). This resulted in a homogeneous and extremely fine oil-in-water emulsion. The emulsion was then transferred to a rotary evaporator (Buchi, Switzerland) and rapidly evaporated to obtain an albumin solution subjected to high pressure homogenization. The evaporator pressure was set during the evaporation by a vacuum pump (Welch) at 1-5 mm Hg and the bath temperature during evaporation was set at 35° C.

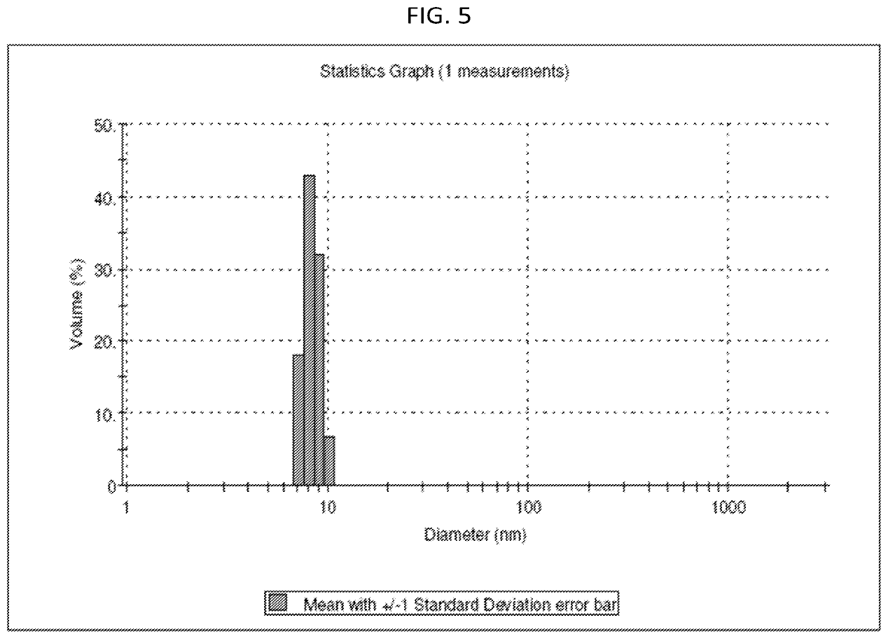

The particle size of the albumin solution was determined by photon correlation spectroscopy with a Malvern Zetasizer. It was observed that there were two peaks, one around 5-8 nm and other around 120-140 nm. The peak around 5-8 nm contained nearly 99% by volume and the peak around 120-140 nm had less than 1% by volume (FIG. 5). As a control, the particle size distribution in 4% human serum solution was measured. It had only one peak around 5-8 nm (FIG. 13). These studies show that the homogenization of an albumin solution in an oil-in-water emulsion renders less than 2-3 percent of the albumin molecules to be aggregated by denaturation.

### Example 2. Preparation of Unstable Solid CBD Nanoparticle without any Inhibitor

An organic solution was prepared by dissolving 602 mg of Cannabidiol (Pur Iso-Labs, LLC, TX, USA) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution was approximately 7.0 and was used without further pH adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 35° C.

An opaque milky white suspension was obtained. The particle size of the suspension was determined by laser diffraction with a Particle Size Analyzer (Beckman Coulter Life Sciences, IN, USA) and found to have formed nanoparticles with a bimodal size distribution between 56 and 110 nm (d₁₀ and d₉₀, respectively) with a d₅₀ size of 79 nm for the first distribution and between 240 and 454 nm (d₁₀ and d₉₀, respectively) with a d₅₀ of 335 nm for the second distribution. The suspension was divided into aliquots and stored at refrigerated and room temperatures; after 24 hours both samples showed a small amount of fine precipitate sedimented on bottom of the containers while remaining an opaque milky white suspension. Particle size analysis of both samples now showed a single distribution; the refrigerated sample showed a size distribution between 411 and 1290 nm (d₁₀ and d₉₀, respectively) with a d₅₀ size of 795 nm and the room temperature sample showed a size distribution between 500 and 2410 nm (d₁₀ and d₉₀) with a d₅₀ of 1240 nm. The d₉₉ after 24 hours for the refrigerated and room temperature samples was 1685 nm and 5120 nm, respectively. The formulation containing the above composition was designated as unstable due to Ostwald ripening and therefore not suitable for sterile filtration.

### Example 3. Preparation of Stable Solid Nanoparticles of Cannabidiol (CBD) with Hexadecyl Hexadecanoate and Cholesterol as Ostwald Ripening Inhibitors

A mixture of 500 mg of Cannabidiol (Pur Iso-Labs, LLC, TX, USA) and 2,499 mg of Hexadecyl hexadecanoate (Spectrum Chemical, NJ, USA), and 126 mg of Cholesterol (Wilshire Technologies, NJ, USA) were dissolved in a mixture of 7.3 mL of Chloroform (Spectrum Chemical, NJ, USA) and 1.2 mL of anhydrous Ethanol (Spectrum Chemical, NJ, USA). A 5% human albumin solution was prepared by diluting 46 mL of 25% human albumin (Grifols Biologicals, Inc., CA, USA) in 105 mL of deionized water (Mueller Water Conditioning, Inc., TX, USA). The pH of the albumin solution was adjusted dropwise with 1N Hydrochloric Acid (Sigma Aldrich Corp., MO, USA) to pH 6.75, determined by a pH meter (Mettler Toledo, OH, USA)

The above organic solution was added to the albumin phase and the mixture was pre-homogenized with a high shear homogenizer at 10,000 RPM (IKA Works, Inc., NC, USA). The crude emulsion was then subjected to high-pressure homogenization (Bee International., MA, USA) at 10,000 psi for 4 passes and 30,000 psi for 12 passes, recycling the emulsion into the process stream after cooling to about 4° C. by passing through a heat exchanger connected to a refrigerated circulator (Temptek, Inc., IN, USA). This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 35 mm Hg, set by a vacuum pump (Leybold USA, Inc., PA, USA), and the bath temperature maintained at 35° C.

An off-white translucent suspension was obtained which was then diluted by 20% volume with water and sufficient sucrose (Spectrum Chemical, NJ, USA) dissolved to give a final diluted concentration of 7% sucrose in the product. The diluted suspension was serially sterile-filtered through 0.45 μm and then 0.22 μm filter units (EMD Millipore, MA, USA). A translucent, slightly hazy yellow, particulate free suspension was obtained. The product was aseptically filled into serum vials and lyophilized (SP Industries, PA, USA) producing a white cake. The particle size of the reconstituted suspension was determined by photon correlation spectroscopy with a Zetasizer (Malvern Panalytical, MA, USA) and found to have formed nanoparticles with a size distribution (intensity based) between 45 and 152 nm (d₁₀ and d₉₀) with a d₅₀ of 83 nm. The suspension was stable at room temperature for up to 3 months.

### Example 4. Preparation of Stable Solid Nanoparticles of Cannabidiol (CBD) with Hexadecyl Hexadecanoate and Cholesterol as Ostwald Ripening Inhibitors

A mixture of 500 mg of Cannabidiol (Pur Iso-Labs, LLC, TX, USA) and 2,499 mg of Hexadecyl hexadecanoate (Spectrum Chemical, NJ, USA), and 126 mg of Cholesterol (Wilshire Technologies, NJ, USA) were dissolved in a mixture of 7.3 mL of Chloroform (Spectrum Chemical, NJ, USA) and 1.2 mL of anhydrous Ethanol (Spectrum Chemical, NJ, USA). A 5% human albumin solution was prepared by diluting 46 mL of 25% human albumin (Grifols Biologicals, Inc., CA, USA) in 105 mL of deionized water (Mueller Water Conditioning, Inc., TX, USA). The pH of the albumin solution was adjusted dropwise with 1N Hydrochloric Acid (Sigma Aldrich Corp., MO, USA) to pH 6.75, determined by a pH meter (Mettler Toledo, OH, USA)

The above organic solution was added to the albumin phase and the mixture was pre-homogenized with a high shear homogenizer at 10,000 RPM (IKA Works, Inc., NC, USA). The crude emulsion was then subjected to high-pressure homogenization (Bee International., MA, USA) at 10,000 psi for 4 passes and 30,000 psi for 12 passes, recycling the emulsion into the process stream after cooling to about 4° C. by passing through a heat exchanger connected to a refrigerated circulator (Temptek, Inc., IN, USA). This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 35 mm Hg, set by a vacuum pump (Leybold USA, Inc., PA, USA), and the bath temperature maintained at 35° C.

An off-white translucent suspension was obtained which was then diluted by 20% volume with water and enough sucrose (Spectrum Chemical, NJ, USA) dissolved to give a final diluted concentration of 7% sucrose in the product. The diluted suspension was serially sterile-filtered through 0.45 μm and then 0.22 μm filter units (EMD Millipore, MA, USA). A translucent, very slightly hazy yellow, particulate free suspension was obtained. The product was aseptically filled into serum vials and lyophilized (SP Industries, PA, USA) producing a white cake. The particle size of the reconstituted suspension was determined by photo correlation spectroscopy with a Zetasizer Nano (Malvern Panalytical, MA, USA) and found to have formed nanoparticles with a size distribution (intensity based) between 45 and 152 nm (d10 and d90) with a d50 of 83 nm. The suspension was stable at room temperature for up to 3 months.

### Example 5. Preparation of Stable Solid Nanoparticles of Cannabidiol (CBD) with Hexadecyl Hexadecanoate and Cholesterol as Ostwald Ripening Inhibitors

A mixture of 158 mg of Cannabidiol (Pur Iso-Labs, LLC, TX, USA), 789 mg of Hexadecyl hexadecanoate (Spectrum Chemical, NJ, USA), and 40 mg of Cholesterol (Wilshire Technologies, NJ, USA) were dissolved in a mixture of 2.7 mL of Chloroform (Spectrum Chemical, NJ, USA) and 0.3 mL of anhydrous Ethanol (Spectrum Chemical, NJ, USA). A 7.5% human albumin solution was prepared by diluting 14.1 mL of 25% human albumin (Grifols Biologicals, Inc., CA, USA) in 32.9 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 25,000 psi for 6 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 29 mm Hg, set by a vacuum pump (Leybold USA, Inc., PA, USA), and the bath temperature maintained at 40° C.

A dark yellow translucent suspension was obtained which was then diluted by 50% volume with 25% human albumin and water for injection to make 5% human albumin in the final product. The diluted suspension was serially sterile-filtered through 0.45 μm and then 0.22 μm filter units (Celltreat Scientific Products, MA, USA). A yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 43 nm with a polydispersity index of 0.195.

### Example 6. Preparation of Stable Solid Nanoparticles of Cannabigerol (CBG) with Hexadecyl Hexadecanoate and Cholesterol as Ostwald Ripening Inhibitors

A mixture of 159 mg of Cannabigerol (Pur Iso-Labs, LLC, TX, USA), 789 mg of Hexadecyl hexadecanoate (Spectrum Chemical, NJ, USA), and 40 mg of Cholesterol (Wilshire Technologies, NJ, USA) were dissolved in a mixture of 2.7 mL of Chloroform (Spectrum Chemical, NJ, USA) and 0.3 mL of anhydrous Ethanol (Spectrum Chemical, NJ, USA). A 7.5% human albumin solution was prepared by diluting 14.1 mL of 25% human albumin (Grifols Biologicals, Inc., CA, USA) in 32.9 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 25,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

A light yellow, slightly translucent suspension was obtained which was then diluted by 50% volume with 25% human albumin and water for injection to make 5% human albumin in the final product. The diluted suspension was serially sterile-filtered through 0.45 μm and then 0.22 μm filter units (Celltreat Scientific Products, MA, USA). A light yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 50 nm with a polydispersity index of 0.230.

### Example 7. Preparation of Stable Solid Nanoparticles of Cannabidiol (CBD) and Cannabigerol (CBG) with Hexadecyl Hexadecanoate and Cholesterol as Ostwald Ripening Inhibitors

A mixture of 80 mg of Cannabidiol (Pur Iso-Labs, LLC, TX, USA), 79 mg of Cannabigerol (Pur Iso-Labs, LLC, TX, USA), 789 mg of Hexadecyl hexadecanoate (Spectrum Chemical, NJ, USA), and 40 mg of Cholesterol (Wilshire Technologies, NJ, USA) were dissolved in a mixture of 2.7 mL of Chloroform (Spectrum Chemical, NJ, USA) and 0.3 mL of anhydrous Ethanol (Spectrum Chemical, NJ, USA). A 7.5% human albumin solution was prepared by diluting 14.1 mL of 25% human albumin (Grifols Biologicals, Inc., CA, USA) in 32.9 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 25,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

A dark yellow, slightly translucent suspension was obtained which was then diluted by 50% volume with 25% human albumin and water for injection to make 5% human albumin in the final product. The diluted suspension was sterile-filtered through a 0.22 μm filter units (Celltreat Scientific Products, MA, USA). A light yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 58 nm with a polydispersity index of 0.269.

### Example 8. Preparation of Stable Solid Nanoparticles of Cannabidiol (CBD) with Hexadecyl Hexadecanoate and Cholesterol as Ostwald Ripening Inhibitors

A mixture of 160 mg of Cannabidiol (Pur Iso-Labs, LLC, TX, USA), 793 mg of Hexadecyl hexadecanoate (Spectrum Chemical, NJ, USA), and 41 mg of Cholesterol (Wilshire Technologies, NJ, USA) were dissolved in a mixture of 2.7 mL of Chloroform (Spectrum Chemical, NJ, USA) and 0.3 mL of anhydrous Ethanol (Spectrum Chemical, NJ, USA). A 7.5% ovalbumin (egg albumin) solution was measured out to 47 mL, having been previously prepared by dissolving 75 mg of ovalbumin (Spectrum Chemical, NJ, USA) per mL of deionized water used (Culligan Water Services, TX, USA) and then serially-filtering until a 0.22 μm filtrate is obtained.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 29 mm Hg, set by a vacuum pump (Leybold USA, Inc., PA, USA), and the bath temperature maintained at 40° C.

An off-white yellow, slightly translucent suspension was obtained which was then serially sterile-filtered without dilution through 1.0 μm, 0.45 μm, and 0.22 μm filter units (Celltreat Scientific Products, MA, USA). An off-white yellow, slightly translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 111 nm with a polydispersity index of 0.130.

### Example 9. Preparation of Stable Solid Nanoparticles of Cannabidiol (CBD) with Hexadecyl Hexadecanoate as the Ostwald Ripening Inhibitor

A mixture of 159 mg of Cannabidiol (Pur Iso-Labs, LLC, TX, USA) and 787 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

An off-white yellow, slightly translucent suspension was obtained which was then serially sterile-filtered without dilution through 1.0 μm and 0.22 μm filter units (Celltreat Scientific Products, MA, USA). An off-white yellow, translucent suspension was obtained. The particle size of the suspension was determined by photo correlation spectroscopy with a Zetasizer Nano (Malvern Panalytical, MA, USA) and found to have formed nanoparticles with a Z-average size of 61 nm with a polydispersity index of 0.170.

### Example 10. Preparation of Stable Solid Nanoparticles of Cannabidiol (CBD) with Hexadecanoic Acid as the Ostwald Ripening Inhibitor

A mixture of 161 mg of Cannabidiol (Pur Iso-Labs, LLC, TX, USA) and 792 mg of Hexadecanoic Acid (MP Biomedicals, LLC, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 30 mm Hg, set by a vacuum pump (Leybold USA, Inc., PA, USA), and the bath temperature maintained at 40° C.

An off-white, milky opaque suspension was obtained which was unable to be sterile-filtered. The particle size of the post-evaporation suspension was determined by photo correlation spectroscopy with a Zetasizer Nano (Malvern Panalytical, MA, USA) and found to have formed particles with a Z-average size of 484 nm with a polydispersity index of 0.360.

### Example 11. Preparation of Stable Solid Nanoparticles of Cannabidiol (CBD) with Hexadecyl Hexadecanoate as the Ostwald Ripening Inhibitor

A mixture of 116 mg of Cannabidiol (Pur Iso-Labs, LLC, TX, USA) and 566 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 28 mm Hg, set by a vacuum pump (Leybold USA, Inc., PA, USA), and the bath temperature maintained at 40° C.

An off-white yellow, very translucent suspension was obtained which was then serially sterile-filtered without dilution through 1.0 μm and 0.22 μm filter units (Celltreat Scientific Products, MA, USA). An off-white yellow, very translucent suspension was obtained. The particle size of the suspension was determined by photo correlation spectroscopy with a Zetasizer Nano (Malvern Panalytical, MA, USA) and found to have formed nanoparticles with a Z-average size of 47 nm with a polydispersity index of 0.176.

### Example 12. Preparation of Stable Solid Nanoparticles of Cannabidiol (CBD) with Hexadecyl Hexadecanoate as the Ostwald Ripening Inhibitor

A mixture of 225 mg of Cannabidiol (Pur Iso-Labs, LLC, TX, USA) and 450 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 29 mm Hg, set by a vacuum pump (Leybold USA, Inc., PA, USA), and the bath temperature maintained at 40° C.

An off-white yellow, slightly translucent suspension was obtained which was then serially sterile-filtered without dilution through 1.0 μm, 0.45 μm, and 0.22 μm filter units (Celltreat Scientific Products, MA, USA). An off-white yellow, translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 47 nm with a polydispersity index of 0.176.

### Example 13. Preparation of Stable Solid Nanoparticles of Cannabidiol (CBD) with Hexadecyl Hexadecanoate as the Ostwald Ripening Inhibitor

A mixture of 341 mg of Cannabidiol (Pur Iso-Labs, LLC, TX, USA) and 341 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 29 mm Hg, set by a vacuum pump (Leybold USA, Inc., PA, USA), and the bath temperature maintained at 40° C.

An off-white yellow, translucent suspension was obtained which was then serially sterile-filtered without dilution through 1.0 μm, 0.45 μm, and 0.22 μm filter units (Celltreat Scientific Products, MA, USA). An off-white yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 50 nm with a polydispersity index of 0.204.

### Example 14. Preparation of Stable Solid Nanoparticles of Cannabidiol (CBD) with Hexadecyl Hexadecanoate and Hydrogenated Soy Phosphatidylcholine as Ostwald Ripening Inhibitors

A mixture of 228 mg of Cannabidiol (Pur Iso-Labs, LLC, TX, USA), 364 mg of Hexadecyl hexadecanoate (Spectrum Chemical, NJ, USA), and 92 mg of Hydrogenated Soy Phosphatidylcholine (Northern Lipids, BC, Canada) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 28 mm Hg, set by a vacuum pump (Leybold USA, Inc., PA, USA), and the bath temperature maintained at 40° C.

An off-white yellow, very translucent suspension was obtained which was then serially sterile-filtered without dilution through 1.0 μm, 0.45 μm, and 0.22 μm filter units (Celltreat Scientific Products, MA, USA). An off-white yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 44 nm with a polydispersity index of 0.210.

### Example 15. Preparation of Stable Solid Nanoparticles of Cannabidiol (CBD) with Hexadecyl Hexadecanoate as the Ostwald Ripening Inhibitor

A mixture of 227 mg of Cannabidiol (Pur Iso-Labs, LLC, TX, USA) and 454 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% bovine albumin solution was prepared by diluting 9.4 mL of 30% bovine serum albumin (Equitech-Bio, Inc., TX, USA) in 37.6 mL of deionized water (Culligan Water Services, TX, USA) and then 0.45 μm filtering (Thermo Scientific Nalgene, MA, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 29 mm Hg, set by a vacuum pump (Leybold USA, Inc., PA, USA), and the bath temperature maintained at 40° C.

A yellow, very translucent suspension was obtained which was then serially sterile-filtered without dilution through 1.0 μm, 0.45 μm, and 0.22 μm filter units (Celltreat Scientific Products, MA, USA). A yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 55 nm with a polydispersity index of 0.184.

### Example 16. Preparation of Stable Solid Nanoparticles of Cannabidiol (CBD) with Hexadecyl Hexadecanoate as the Ostwald Ripening Inhibitor

A mixture of 339 mg of Cannabidiol (Pur Iso-Labs, LLC, TX, USA) and 339 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 7.5% ovalbumin (egg albumin) solution was measured out to 47 mL, having been previously prepared by dissolving 75 mg of ovalbumin (Spectrum Chemical, NJ, USA) per mL of deionized water used (Culligan Water Services, TX, USA) and then serially-filtering until a 0.22 μm filtrate is obtained.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 12 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 40° C.

An off-white yellow, slightly translucent suspension was obtained which was then serially sterile-filtered without dilution through 1.0 μm, 0.45 μm, and 0.22 μm filter units (Celltreat Scientific Products, MA, USA). An off-white yellow, slightly translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 69 nm with a polydispersity index of 0.094.

### Example 17. Preparation of Stable Solid Nanoparticles of Cannabidiol (CBD) with Hexadecyl Hexadecanoate as the Ostwald Ripening Inhibitor

A mixture of 339 mg of Cannabidiol (Pur Iso-Labs, LLC, TX, USA) and 341 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% ovalbumin (egg albumin) solution was measured out to 47 mL, having been previously prepared by dissolving 75 mg of ovalbumin (Spectrum Chemical, NJ, USA) per mL of deionized water used (Culligan Water Services, TX, USA), then serially-filtering until a 0.22 μm filtrate is obtained, and for every 100 mL required, diluting 66.7 mL of 7.5% ovalbumin with 33.3 mL of deionized water.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 12 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 40° C.

An off-white yellow, slightly translucent suspension was obtained which was then serially sterile-filtered without dilution through 1.0 μm, 0.45 μm, and 0.22 μm filter units (Celltreat Scientific Products, MA, USA). An off-white yellow, slightly translucent suspension was obtained. The particle size of the suspension was determined by photo correlation spectroscopy with a Zetasizer Nano (Malvern Panalytical, MA, USA) and found to have formed nanoparticles with a Z-average size of 70 nm with a polydispersity index of 0.115.

### Example 18. Preparation of Stable Solid Nanoparticles of Cannabidiol (CBD) with Hexadecyl Hexadecanoate as the Ostwald Ripening Inhibitor

A mixture of 340 mg of Cannabidiol (Pur Iso-Labs, LLC, TX, USA) and 341 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 7.5% ovalbumin (egg albumin) solution was measured out to 47 mL, having been previously prepared by dissolving 75 mg of ovalbumin (Spectrum Chemical, NJ, USA) per mL of deionized water used (Culligan Water Services, TX, USA) then serially-filtering until a 0.22 μm filtrate is obtained, and for every 100 mL required, diluting 66.7 mL of 7.5% ovalbumin with 33.3 mL of deionized water.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 25,000 psi for 12 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 23 mm Hg, set by a vacuum pump (Leybold USA, Inc., PA, USA), and the bath temperature maintained at 40° C.

An off-white yellow, slightly translucent suspension was obtained which was then serially sterile-filtered without dilution through 1.0 μm, 0.45 μm, and 0.22 μm filter units (Celltreat Scientific Products, MA, USA). An off-white yellow, slightly translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 69 nm with a polydispersity index of 0.090.

### Example 19. Preparation of Stable Solid Nanoparticles of Cannabidiol (CBD) with Hexadecyl Hexadecanoate and Soy Lecithin as Ostwald Ripening Inhibitors

A mixture of 229 mg of Cannabidiol (Pur Iso-Labs, LLC, TX, USA), 362 mg of Hexadecyl hexadecanoate (Spectrum Chemical, NJ, USA), and 89 mg of Soy Lecithin (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

An off-white yellow, very translucent suspension was obtained which was then sterile-filtered without dilution through a 0.22 μm filter units (Celltreat Scientific Products, MA, USA). An off-white yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 43 nm with a polydispersity index of 0.202.

### Example 20. Preparation of Concentrated Stable Solid Nanoparticles of Cannabidiol (CBD) with Hexadecyl Hexadecanoate as Ostwald Ripening Inhibitor

A mixture of 676 mg of Cannabidiol (Cope, CO, USA) and 676 mg of Hexadecyl hexadecanoate (Spectrum Chemical, NJ, USA) were dissolved in a mixture of 5.4 mL of Chloroform (Spectrum Chemical, NJ, USA) and 0.6 mL of anhydrous Ethanol (Spectrum Chemical, NJ, USA). A 5% human albumin solution was prepared by diluting 18.8 mL of 25% human albumin (Grifols Biologicals, Inc., CA, USA) in 75.2 mL of Water for Injection (Rocky Mountain Biologicals, UT, USA).

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion, with half the volume that was collected being transferred at once to a rotary evaporator (Yamato Scientific America, Inc., CA, USA) and rapidly evaporated to a nanoparticle suspension at an initial pressure of 28 mm Hg, set by a vacuum pump (Leybold USA, Inc., PA, USA), and the bath temperature maintained at 40° C. The suspension was removed from the flask, the flask quickly cleaned and replaced on the rotovap and evacuated. The remaining volume of emulsion had remained stable for approximately 10 minutes before being rapidly evaporated. Both evaporation products produced an off-white yellow, very translucent suspension that were combined back into the rotary flask and gently evaporated until the volume had reduced by about 80%.

A dark yellow, almost brown translucent suspension was obtained which was then sterile-filtered without dilution through a 0.22 μm filter units (Celltreat Scientific Products, MA, USA). A dark yellow, almost brown translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 49 nm with a polydispersity index of 0.200.

### Example 21. Preparation of Stable Solid Nanoparticles of Cannabidiol (CBD) with Hexadecyl Hexadecanoate as Ostwald Ripening Inhibitors and Alpha-Lactalbumin as Protein

A mixture of 340 mg of Cannabidiol (Cope, CO, USA) and 338 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 7.5% alpha-Lactalbumin solution was prepared by dissolving 3.53 g of alpha-Lactalbumin powder (Agropur, WI, USA) in about 40 mL of Water for Injection (Rocky Mountain Biologicals, UT, USA) and then adjusting the final volume to 47 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 20 mm Hg, set by a vacuum pump (Leybold USA, Inc., PA, USA), and the bath temperature maintained at 40° C.

An off-white pale yellow, very translucent suspension was obtained which was then sterile-filtered without dilution through a 0.22 μm filter units (Celltreat Scientific Products, MA, USA). An off-white pale yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 41 nm with a polydispersity index of 0.163.

### Example 22. Preparation of Stable Solid Nanoparticles of Cannabidiol (CBD) with Hexadecyl Hexadecanoate as Ostwald Ripening Inhibitors and Water Soluble Soy Proteins (“Aquafaba”)

A mixture of 341 mg of Cannabidiol (Cope, CO, USA) and 339 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 16 oz can of food grade organic chickpeas was obtained and the entire liquid solution covering the contents of the can (colloquially known as “Aquafaba”) was removed and serially sterile filtered through 0.45 and 0.22 μm filter units (Celltreat Scientific Products, MA, USA) to give a yellow, slightly translucent solution that was used without further dilution.

The above organic solution was added to the 47 mL of aqueous phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 20 mm Hg, set by a vacuum pump (Leybold USA, Inc., PA, USA), and the bath temperature maintained at 40° C.

An off-white milky, very slightly translucent suspension was obtained which was then sterile-filtered without dilution through 0.45 and 0.22 μm filter units (Celltreat Scientific Products, MA, USA). An off-white milky, very slightly translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 134 nm with a polydispersity index of 0.168.

### Example 23. Preparation of Stable Solid Nanoparticles of Cannabidiol (CBD) with Hexadecyl Hexadecanoate as Ostwald Ripening Inhibitor and Alpha-Lactalbumin as Protein

A mixture of 341 mg of Cannabidiol (Cope, CO, USA) and 339 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% alpha-Lactalbumin solution was prepared by dissolving 2.35 g of alpha-Lactalbumin powder (Agropur, WI, USA) in about 40 mL of Water for Injection (Rocky Mountain Biologicals, UT, USA) and then adjusting the final volume to 47 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

A very light pale yellow, very translucent suspension was obtained which was then sterile-filtered without dilution through a 0.22 μm filter units (Celltreat Scientific Products, MA, USA). A very light pale yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 42 nm with a polydispersity index of 0.160.

### Example 24. Preparation of Stable Solid Nanoparticles of 5:1 Cannabidiol (CBD) with Hexadecyl Hexadecanoate as Ostwald Ripening Inhibitor and Alpha-Lactalbumin as Protein

A mixture of 565 mg of Cannabidiol (Cope, CO, USA) and 113 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% alpha-Lactalbumin solution was prepared by dissolving 2.35 g of alpha-Lactalbumin powder (Agropur, WI, USA) in about 40 mL of Water for Injection (Rocky Mountain Biologicals, UT, USA) and then adjusting the final volume to 47 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 25 mm Hg, set by a vacuum pump (Leybold USA, Inc., PA, USA), and the bath temperature maintained at 40° C.

An off-white, slightly translucent suspension was obtained which was then sterile-filtered without dilution through a 0.22 μm filter units (Celltreat Scientific Products, MA, USA). An off-white slightly translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 108 nm with a polydispersity index of 0.246.

### Example 25. Preparation of Stable Solid Nanoparticles of Cannabidiol (CBD) with Ubiquinone (Coenzyme Q10) as Ostwald Ripening Inhibitor and Alpha-Lactalbumin as Protein

A mixture of 340 mg of Cannabidiol (Cope, CO, USA) and 339 mg of Ubiquinone (Coenyzme Q10, PureBulk.com, OR, USA) were dissolved in a mixture of 2.7 mL of Chloroform (Spectrum Chemical, NJ, USA) and 0.3 mL of anhydrous Ethanol (Spectrum Chemical, NJ, USA). A 5% alpha-Lactalbumin solution was prepared by dissolving 2.35 g of alpha-Lactalbumin powder (Agropur, WI, USA) in about 40 mL of Water for Injection (Rocky Mountain Biologicals, UT, USA) and then adjusting the final volume to 47 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 20 mm Hg, set by a vacuum pump (Leybold USA, Inc., PA, USA), and the bath temperature maintained at 40° C.

A light orange, very translucent suspension was obtained which was then sterile-filtered without dilution through a 0.22 μm filter units (Celltreat Scientific Products, MA, USA). A light orange, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 47 nm with a polydispersity index of 0.166.

### Example 26. Preparation of Stable Solid Nanoparticles of Cannabidiol (CBD) with Hexadecyl Hexadecanoate as Ostwald Ripening Inhibitor and Alpha-Lactalbumin as Protein and Processed with Cannabinoid Type Terpenes

A mixture of 337 mg of Cannabidiol (Cope, CO, USA), 342 mg of Hexadecyl hexadecanoate (Spectrum Chemical, NJ, USA), and 50 μL of a Terpene mixture simulating common terpenes found in Cannabis sativa, all food grade components (Sigma Aldrich, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% alpha-Lactalbumin solution was prepared by dissolving 2.35 g of alpha-Lactalbumin powder (Agropur, WI, USA) in about 40 mL of Water for Injection (Rocky Mountain Biologicals, UT, USA) and then adjusting the final volume to 47 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 21 mm Hg, set by a vacuum pump (Leybold USA, Inc., PA, USA), and the bath temperature maintained at 40° C.

A pale white yellow, very translucent suspension was obtained which was then sterile-filtered without dilution through a 0.22 μm filter units (Celltreat Scientific Products, MA, USA). A pale white yellow, very translucent to clear, particulate-free suspension was obtained with a significant floral odor, very similar to the starting terpene mixture added. The particle size of the suspension was determined by photo correlation spectroscopy with a Zetasizer Nano (Malvern Panalytical, MA, USA) and found to have formed nanoparticles with a Z-average size of 45 nm with a polydispersity index of 0.154.

### Example 27. Preparation of Stable Solid Nanoparticles of Cannabidiol (CBD) with Hexadecyl Hexadecanoate as Ostwald Ripening Inhibitor and Alpha-Lactalbumin as Protein

A mixture of 338 mg of Cannabidiol Broad Spectrum Distillate (Kadenwood Biosciences, CA, USA—distillate is composed of 94.64% CBD, 1.98% CBG, 0.85% CBDV, 0.03% CBN, and 0.07% D9-THC totaling 97.58% phytocannabinoids) and 339 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% alpha-Lactalbumin solution was prepared by dissolving 2.35 g of alpha-Lactalbumin powder (Agropur, WI, USA) in about 40 mL of Water for Injection (Rocky Mountain Biologicals, UT, USA) and then adjusting the final volume to 47 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 20 mm Hg, set by a vacuum pump (Leybold USA, Inc., PA, USA), and the bath temperature maintained at 40° C.

A pale white yellow, very translucent to clear, particulate-free suspension was obtained without filtration. The particle size of the unfiltered suspension was determined by photo correlation spectroscopy with a Zetasizer Nano (Malvern Panalytical, MA, USA) and found to have formed nanoparticles with a Z-average size of 42 nm with a polydispersity index of 0.156.

### Example 28. Preparation of Stable Solid Nanoparticles of Cannabinol (CBN) with Hexadecyl Hexadecanoate as Ostwald Ripening Inhibitor and Alpha-Lactalbumin as Protein

A mixture of 340 mg of Cannabinol (Pure-Iso Labs, TX, USA) and 339 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 5% alpha-Lactalbumin solution was prepared by dissolving 2.35 g of alpha-Lactalbumin powder (Agropur, WI, USA) in about 40 mL of Water for Injection (Rocky Mountain Biologicals, UT, USA) and then adjusting the final volume to 47 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 22 mm Hg, set by a vacuum pump (Leybold USA, Inc., PA, USA), and the bath temperature maintained at 40° C.

A pale white yellow, very translucent, particulate-free suspension was obtained without filtration. The particle size of the unfiltered suspension was determined by photo correlation spectroscopy with a Zetasizer Nano (Malvern Panalytical, MA, USA) and found to have formed nanoparticles with a Z-average size of 43 nm with a polydispersity index of 0.155.

### Example 29. Preparation of Stable Solid Nanoparticles of Cannabidiol (CBD) with Hexadecyl Hexadecanoate as Ostwald Ripening Inhibitor and Alpha-Lactalbumin as Protein

A mixture of 339 mg of Cannabidiol (Kadenwood Biosciences, CA, USA) and 338 mg of Hexadecyl hexadecanoate (Spectrum Chemical, NJ, USA) were dissolved in a mixture of 2.7 mL of Chloroform (Spectrum Chemical, NJ, USA) and 0.3 mL of anhydrous Ethanol (Spectrum Chemical, NJ, USA). A 1% alpha-Lactalbumin solution was prepared by dissolving 0.47 g of alpha-Lactalbumin powder (Agropur, WI, USA) in about 40 mL of Water for Injection (Rocky Mountain Biologicals, UT, USA) and then adjusting the final volume to 47 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 23 mm Hg, set by a vacuum pump (Leybold USA, Inc., PA, USA), and the bath temperature maintained at 40° C.

A pale off white, very translucent, particulate-free suspension was obtained without filtration. The particle size of the unfiltered suspension was determined by photo correlation spectroscopy with a Zetasizer Nano (Malvern Panalytical, MA, USA) and found to have formed nanoparticles with a Z-average size of 50 nm with a polydispersity index of 0.186.

### Example 30. Preparation of Stable Solid Nanoparticles of Cannabidiol (CBD) with Hexadecyl Hexadecanoate as Ostwald Ripening Inhibitor and Alpha-Lactalbumin as Protein

A mixture of 1193 mg of Cannabidiol (Cope, CO, USA) and 910 mg of Hexadecyl hexadecanoate (Spectrum Chemical, NJ, USA) were dissolved in a mixture of 6.3 mL of Chloroform (Spectrum Chemical, NJ, USA) and 0.7 mL of anhydrous Ethanol (Spectrum Chemical, NJ, USA). A 5% alpha-Lactalbumin solution was prepared by dissolving 2.15 g of alpha-Lactalbumin powder (Agropur, WI, USA) in about 30 mL of Water for Injection (Rocky Mountain Biologicals, UT, USA) and then adjusting the final volume to 43 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 22 mm Hg, set by a vacuum pump (Leybold USA, Inc., PA, USA), and the bath temperature maintained at 40° C.

A pale white yellow, translucent, particulate-free suspension was obtained without filtration. The particle size of the unfiltered suspension was determined by photo correlation spectroscopy with a Zetasizer Nano (Malvern Panalytical, MA, USA) and found to have formed nanoparticles with a Z-average size of 49 nm with a polydispersity index of 0.175.

### Example 31. Preparation of Stable Solid Nanoparticles of Cannabidiol (CBD) and Cannabinol (CBN) with Hexadecyl Hexadecanoate as Ostwald Ripening Inhibitor and Alpha-Lactalbumin as Protein

A mixture of 1429 mg of Cannabidiol (Cope, CO, USA), 1427 mg of Cannabinol (Pure-Iso Labs, TX, USA) and 2819 mg of Hexadecyl hexadecanoate (Spectrum Chemical, NJ, USA) were dissolved in a mixture of 15.12 mL of Chloroform (Spectrum Chemical, NJ, USA) and 1.68 mL of anhydrous Ethanol (Spectrum Chemical, NJ, USA). A 5% alpha-Lactalbumin solution was prepared by dissolving 5.15 g of alpha-Lactalbumin powder (Agropur, WI, USA) in about 80 mL of Water for Injection (Rocky Mountain Biologicals, UT, USA) and then adjusting the final volume to 103 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 40° C.

A pale off white, slightly translucent suspension was obtained without filtration. The particle size of the unfiltered suspension was determined by photo correlation spectroscopy with a Zetasizer Nano (Malvern Panalytical, MA, USA) and found to have formed nanoparticles with a Z-average size of 46 nm with a polydispersity index of 0.177.

### Example 32. Preparation of Stable Solid Nanoparticles of Delta 8 Tetrahydrocannabinol (Δ8-THC) with Hexadecyl Hexadecanoate as Ostwald Ripening Inhibitor and Alpha-Lactalbumin as Protein

A mixture of 2825 mg of hemp derived A8-Tetrahydrocannabinol (Pure-Iso Labs, TX, USA) and 2183 mg of Hexadecyl hexadecanoate (Spectrum Chemical, NJ, USA) were dissolved in a mixture of 15.12 mL of Chloroform (Spectrum Chemical, NJ, USA) and 1.68 mL of anhydrous Ethanol (Spectrum Chemical, NJ, USA). A 5% alpha-Lactalbumin solution was prepared by dissolving 5.15 g of alpha-Lactalbumin powder (Agropur, WI, USA) in about 80 mL of Water for Injection (Rocky Mountain Biologicals, UT, USA) and then adjusting the final volume to 103 mL with WFI.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 21 mm Hg, set by a vacuum pump (Leybold USA, Inc., PA, USA), and the bath temperature maintained at 40° C.

A pale yellow, very translucent suspension was obtained which was then sterile-filtered without dilution through a 0.22 μm filter units (Celltreat Scientific Products, MA, USA). A pale yellow, very translucent to clear, particulate-free suspension was obtained. The particle size of the suspension was determined by photo correlation spectroscopy with a Zetasizer Nano (Malvern Panalytical, MA, USA) and found to have formed nanoparticles with a Z-average size of 43 nm with a polydispersity index of 0.171.

### Example 33. Preparation of Quickset Gelatin “Gummy” Candies with Stable Solid Nanoparticles of Cannabidiol (CBD)

A “gummy” base solution was made by dissolving 56 g of commercially available flavored gelatin mix and 1.16 g of unflavored gelatin into 25.1 g of water with microwave heating. The solution was allowed to rest and a “scum” layer rose to the surface and was removed to leave a clear viscous liquid base. To 48 mL of this base was slowly added with gentle stirring 16 mL of CBD Nanoparticles suspension, which produced a translucent, less viscous solution which was then aliquoted as 4 mL units (equivalent to 1 mL of the nano suspension in each gummy) into a lightly oiled (unflavored cooking spray) silicone mold and placed in a refrigerator for 1 hour. The gummies were easily removed intact from the mold and allowed to rest uncovered in refrigerator for at least 24 hours to firm up.

A small slice of a gummy unit was placed into a test tube and then a few mL of deionized water was added, then the test tube placed in a water bath at 70° C. and the gummy piece began to immediately dissolve to give a translucent solution. An aliquot of this was 5.0 μm syringe filtered into a cuvette and then the particle size determined by photo correlation spectroscopy with a Zetasizer Nano (Malvern Panalytical, MA, USA) and found to have a Z-average size of 417 nm with a polydispersity index of 0.248. When an experiment was performed to produce a placebo gummy candy and 2 gummies made with NIST Polystyrene latex size standards, (60 and 80 nm, Thermo Fisher Scientific, MA, USA), the results show no size distribution in the placebo gummy, and showed 460 and 480 nm size distributions after 0.45 μm filtering and sizing the samples, showing the measured size increase is due to viscosity increasing from the gelatin present in the sample, and inferring that the nanoparticles are present in the gummies.

Several persons who previously tried the CBD nanoparticle suspension (1 mL, equivalent to approximately 25 mg CBD) and experienced a strong response, also reported a similar response when taking a single gummy, with an average of 30-40 minutes of onset time and the effects lasting strongly for 3-5 hours for either form (liquid suspension or gummy).

### Example 34. Preparation of Commercial Formulation Pectin “Gummy” Candies with Stable Solid Nanoparticles

A sample of CBD Nanoparticles suspension was provided to Virc LLC, and gummy candy Contract Manufacturing Organization who created a “lab batch” of our CBD nanoparticles into a pectin based gummy using a proprietary formulation that uses the following ingredients: Organic cane sugar, glucose syrup, pectin (from fruit), citric acid, sodium citrate, natural tangerine and orange with other natural flavors, ascorbic acid, potassium citrate, sea salt, beta carotene (from carrots), carnauba leaf wax.

A slice of a sample gummy was dissolved in hot water in a test tube and then an aliquot was 0.45 μm filtered into a cuvette then the particle size determined by photo correlation spectroscopy with a Zetasizer Nano (Malvern Panalytical, MA, USA) and found to have a Z-average size of 486 nm with a polydispersity index of 0.195.

Several persons who previously tried the CBD nanoparticle suspension (1 mL, equivalent to approximately 25 mg CBD) and Quickset Gelatin gummies that experienced a strong response, also reported a similar response when taking an equivalent commercial pectin gummy.

### Example 35. Preparation of Quickset Gelatin “Gummy” Candies with Stable Solid Nanoparticles of Δ8-THC

A “gummy” base solution was made by dissolving 56 g of commercially available flavored gelatin mix and 1.16 g of unflavored gelatin into 25.1 g of water with microwave heating. The solution was allowed to rest and a “scum” layer rose to the surface and was removed to leave a clear viscous liquid base. To 48 mL of this base was slowly added with gentle stirring 6.4 mL of Δ8-THC Nanoparticles suspension, which produced a translucent, less viscous solution which was then aliquoted as 4 mL units (equivalent to 1 mL of the nano suspension in each gummy) into a lightly oiled (unflavored cooking spray) silicone mold and placed in a refrigerator for 1 hour. The gummies were easily removed intact from the mold and allowed to rest uncovered in refrigerator for at least 24 hours to firm up.

A small slice of a gummy unit was placed into a test tube and then a few mL of deionized water was added, then the test tube placed in a water bath at 70° C. and the gummy piece began to immediately dissolve to give a translucent solution. An aliquot of this was 5.0 μm syringe filtered into a cuvette and then the particle size determined by photo correlation spectroscopy with a Zetasizer Nano (Malvern Panalytical, MA, USA) and found to have a Z-average size of 417 nm with a polydispersity index of 0.248. When an experiment was performed to produce a placebo gummy candy and 2 gummies made with NIST Polystyrene latex size standards, (60 and 80 nm, Thermo Fisher Scientific, MA, USA), the results show no size distribution in the placebo gummy, and showed 460 and 480 nm size distributions after 0.45 μm filtering and sizing the samples, showing the measured size increase is due to viscosity increasing from the gelatin present in the sample, and inferring that the nanoparticles are present in the gummies.

Several persons who previously tried the Δ8-THC nanoparticle suspension (0.4 mL, equivalent to approximately 10 mg Δ8-THC) and experienced a strong response within 3-4 minutes, also reported a similar response when taking a single gummy, with an average of 30-40 minutes of onset time and the effects lasting strongly for 3-5 hours for either form (liquid suspension or gummy).

1. A pharmaceutical composition comprising a substantially stable and sterile filterable dispersion of solid nanoparticles in an aqueous medium, wherein the solid nanoparticles comprise a substantially water insoluble pharmaceutically active substance or mixture thereof, and have a mean particle size of less than 220 nm as meaured by photon correlation spectroscopy, wherein the substantially water insoluble pharmaceutically active substance comprises a cannabinoid and/or cannabinoid analog, wherein the composition is prepared by a process comprising:
(a) combining an aqueous phase comprising water and a biocompatible polymer as emulsifier and an organic phase comprising the substantially water insoluble cannabinoids and cannabinoid analogs, a water-immiscible organic solvent, optionally a water-miscible organic solvent as an interfacial lubricant and at least one Ostwald ripening inhibitor;
(b) forming an oil-in-water emulsion using a high-pressure homogenizer;
(c) removing the water-immiscible organic solvent and the water-miscible organic solvent from the oil-in water emulsion under vacuum, thereby forming a substantially stable dispersion of solid nanoparticles comprising the Ostwald ripening inhibitor, the biocompatible polymeric emulsifier and the substantially water insoluble cannabinoids and cannabinoid analogs in the aqueous medium; wherein
(i) the Ostwald ripening inhibitor is a non-polymeric hydrophobic organic compound that is substantially insoluble in water;
(ii) the Ostwald ripening inhibitor is less soluble in water than the substantially water insoluble cannabinoids and cannabinoid analogs;
(iii) the Ostwald ripening inhibitor is selected from the group consisting of:
(a) a mono-, di- or a ti-glyceride of a fatty acid;
(b) a fatty acid mono- or di-ester of a C₂₋₁₀ diol;
(c) a fatty acid ester of an alkanol or a cycloalkanoyl;
(d) a wax;
(e) a long chain aliphatic alcohol;
(f) a hydrogenated vegetable oil;
(g) cholesterol or fatty acid ester of cholesterol;
(h) a ceramide;
(i) a coenzyme Q10;
(j) a lipoic acid or an ester of lipoic acid;
(k) a phospholipid in an amount insufficient to form vesicles; and
(l) combinations thereof.

2. The pharmaceutical composition, according to claim 1, wherein the substantially water insoluble pharmaceutically active substance is a cannabinoid or cannabinoid analog and is selected from the group consisting of plant derived tetrahydrocannabidiol (THC), synthetic tetrahydrocannabinol (THC or Dronabinol), plant derived cannabidiol (CBD), synthetic CBD, nabilone, HU-210, dexanabinol, Cannabicyclol (CBL), Cannabigerol (CBG) and Cannabichromene (CBC), Cannabielsoin (CBE) and Cannabinodiol (CBND), cannabinol (CBN), tetrahydrocannabinolic acid (THCA) and cannabidivarine (CBDV) and combinations thereof.

3. The pharmaceutical composition, according to claim 1, is suitable for the treatment of epilepsy/seizure, pain, nausea and vomiting, anorexia, anti-psoriatic, antipsychotic, anti-proliferative, anti-emetic, anti-inflammatory, anti-diabetic, antibacterial, antispasmdic, anorectic, anti-insomnia, anti-ischemic, antifungal, antibacterial, intestinal anti-prokinetic, immunosuppressive, bone stimulant, Alzheimer's, anxiety, atherosclerosis, arthritis cancer, peripheral neurophathy, colitis/Crohn's, depression, fibromyalgia, glaucoma, irritable bowel, multiple sclerosis, neurodegeneration, obesity, osteoporosis, Parkinson's, PTSD, schizophrenia, substance dependence/addiction, and stroke/traumatic brain injury.

4. The pharmaceutical composition, according to claim 1, wherein the Ostwald ripening inhibitor or mixture thereof, is sufficiently miscible with the water-insoluble drug to form solid particles in the dispersion, wherein the particles comprise a substantially single-phase mixture of the water insoluble drug and the Ostwald ripening inhibitor or mixture thereof.

5. The pharmaceutical composition, according to claim 1, wherein said biocompatible polymer is human albumin or recombinant human albumin or PEG-human albumin or bovine serum albumin or the like.

6. The pharmaceutical composition, according to claim 1, is admininistered by oral, inhalation, nasal and parenteral routes.

7. The pharmaceutical composition, according to claim 1, further comprising pharmaceutically acceptable preservative or mixture thereof, wherein said preservative is selected from the group consisting of phenol, chlorobutanol, benzylalcohol, methylparaben, propylparaben, benzalkonium chloride and cetylpyridinium chloride.

8. The pharmaceutical composition, according to claim 1, further comprising a biocompatible chelating agent wherein said biocompatible chelating agent is selected from the group consisting of ethylenediaminetetraacetic acid (EDTA), diethylenetriaminepentaacetic acid (DTPA), ethylene glycol-bis(O-aminoethyl ether)-tetraacetic acid (EGTA), N (hydroxyethyl) ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.

9. (canceled)

10. (canceled)

11. The pharmaceutical composition, according to claim 1, further comprising a cryoprotectant/bulking agent selected from the group consisting of mannitol, sucrose and trehalose.

12. The pharmaceutical composition, according to claim 1, wherein the weight fraction of Ostwald ripening inhibitor relative to the total weight of water insoluble drug is from 0.01 to 0.99.

13. The pharmaceutical composition, according to claim 1, wherein the aqueous medium containing the solid nanoparticle is sterilized by filtering through a 0.22-micron filter.

14. The pharmaceutical composition in claim 13, wherein the pharmaceutical composition is spray-dried or freeze-dried or lyophilized.

15. A composition comprising a substantially stable dispersion of solid nanoparticles in an aqueous medium,
wherein the solid nanoparticles comprise
i) a cannabinoid and/or a cannabinoid analog; and
ii) at least one Ostwald ripening inhibitor.

16. The composition of claim 15, wherein the composition is sterile filterable and the nanoparticles have a mean particle size of less than 220 nm as measured by photon correlation spectroscopy.

17. The composition of claim 15, wherein the composition further comprises a biocompatible polymer as emulsifier.

18. The composition of claim 15, wherein the Ostwald ripening inhibitor is selected from the group consisting of:
(a) a mono-, di- or a ti-glyceride of a fatty acid;
(b) a fatty acid mono- or di-ester of a C₂₋₁₀ diol;
(c) a fatty acid ester of an alkanol or a cycloalkanoyl;
(d) a wax;
(e) a long chain aliphatic alcohol;
(f) a hydrogenated vegetable oil;
(g) cholesterol or fatty acid ester of cholesterol;
(h) a ceramide;
(i) a coenzyme Q10;
(j) a lipoic acid or an ester of lipoic acid;
(k) a phospholipid in an amount insufficient to form vesicles; and
(l) combinations thereof.

19. The composition of claim 15, wherein the cannabinoid or cannabinoid analog and is selected from the group consisting of plant derived tetrahydrocannabidiol (THC), synthetic tetrahydrocannabinol (THC or Dronabinol), plant derived cannabidiol (CBD), synthetic CBD, nabilone, HU-210, dexanabinol, Cannabicyclol (CBL), Cannabigerol (CBG) and Cannabichromene (CBC), Cannabielsoin (CBE) and Cannabinodiol (CBND), cannabinol (CBN), tetrahydrocannabinolic acid (THCA) and cannabidivarine (CBDV) and combinations thereof.

20. The composition of claim 15, wherein the Ostwald ripening inhibitor or mixture thereof, is sufficiently miscible with the cannabinoid or cannabinoid analog to form solid particles in the dispersion, wherein the particles comprise a substantially single-phase mixture of the cannabinoid or cannabinoid analog and the Ostwald ripening inhibitor or mixture thereof.

21. The composition of claim 16, wherein said biocompatible polymer is human albumin or recombinant human albumin or PEG-human albumin or bovine serum albumin.

22. A method of treating a disease or condition in a subject, comprising administering to the subject an effective amount of the composition of claim 1.
